(19) United States
(12) Patent Application Publication
(10) Pub. No.: US 2015/0017210 A1
(43) Pub. Date: Jan. 15, 2015

(54) Gene Signature Predicts Adenocarcinoma Prognosis and Therapeutic Response

(71) Applicant: Board of Regents, The University of Texas System, Austin, TX (US)

(72) Inventors: Yang XIE, Coppell, TX (US); Hao TANG, Dallas, TX (US); Guanghua XIAO, Coppell, TX (US); Ignacio WISTUBA, Houston, TX (US); John D. MINNA, Dallas, TX (US)

(21) Appl. No.: 14/316,293

(22) Filed: Jun. 26, 2014

Related U.S. Application Data
(60) Provisional application No. 61/844,002, filed on Jul. 9, 2013.

(51) Int. Cl. C12Q 1/68; G01N 33/574

(52) U.S. Cl. CPC C12Q 1/6886; G01N 33/57423; C12Q 2600/118; C12Q 2600/158; C12Q 2600/106
USPC 424/278.1; 506/9; 424/649; 514/449; 600/1

(57) ABSTRACT

The present invention is drawn to methods of predicting prognosis of, and therapeutic response in, carcinoma of the lung.

This application claims benefit of priority to U.S. Provisional Application Ser. No. 61/844,022, filed Jul. 9, 2013, the entire contents of which are hereby incorporated by reference.

The invention was made with government support under grant no. 1R01CA152301, awarded by the National Institutes of Health. The government has certain rights in the invention.

### BACKGROUND OF THE INVENTION

I. Field of the Invention

The present invention relates generally to the fields of genetics, molecular biology, and oncology. In certain aspects, the invention is related to to use of a panel of genes whose disregulated expression is prognostic certain cancers as well as the response to therapy.

II. Description of Related Art

Lung cancer is the leading cause of cancer-related mortality worldwide (Jemal et al., 2008). Even after an apparent complete resection of non-small-cell lung cancer (NSCLC), 33% of patients with pathologic stage IA and 77% with stage IIIA disease die within 5 years of diagnosis. Several randomized trials have demonstrated that there is a survival benefit with adjuvant chemotherapy (ACT) in resected NSCLC (Douillard et al., 2006; Kato et al., 2004; The International Adjuvant Lung Cancer Trial Collaborative Group, 2004; Winton et al., 2005 and Strauss et al., 2008). However, the effect is modest—only 4-15% improvement in 5-year survival, while at the same time, such treatment may cause serious adverse effects (Winton et al., 2005 and Olaussen et al., 2007). Because the response to standard chemotherapy in lung cancer varies, it would be very helpful to prospectively identify the subgroup(s) of patients who are unlikely to benefit from ACT, and therefore, can be spared the effects of unnecessary treatment.

Recently, several groups have developed gene expression signatures aiming to classify lung cancer patients into groups with distinct clinical outcomes (Chen et al., 2011; Chen et al., 2007; Lee et al., 2008; Lu et al., 2006; Navab et al., 2011; Shedden et al., 2008; Tomida et al., 2004; Wigle et al., 2002; Xie et al., 2011; Zhu et al., 2010; Jeong et al., 2010; Kratz et al., 2012; Boutros et al., 2009 and Roepman et al., 2009). However, most current molecular signatures for lung cancer are prognostic only, and do not provide any estimation as to whether a patient would benefit from ACT. In addition, the signatures often contain large numbers of genes, with limited information about the functional importance of the genes. All of these problems limit the clinical application of those signatures.

### SUMMARY OF THE INVENTION

Thus, in accordance with the present invention, there is provided a method of predicting the survival of a human subject diagnosed with adenocarcinoma tumor of the lung comprising obtaining expression information for 9 or more of the following genes in a lung adenocarcinoma cancer sample obtained from said subject:
RPM2, ARUKA, CDKN3, PRC1, HOPX, DPP4, ATP8A1, CYP2B6, DOCK9, COL4A3, Clorf116, TTC37, IFT57, HSD17B6, NKX2-1, GPR116, MBIP, and/or SLC35A5,
   wherein an alteration in the expression 9 or more of said genes, as compared to the average expression in an adenocarcinoma of the lung, indicates that said subject has a worse than average survival. The decrease and/or increase of expression may be at least 0.2-fold. The method may further comprise treating said patient with an aggressive therapy if predicted to have a worse than average prognosis for survival. The aggressive therapy comprises chemotherapy, such a platin compound and/or a taxane compound, and/or radiotherapy. The method may further comprising obtaining expression information for 10, 11, 12, 13, 14, 15, 16, 17, or all 18 of said genes. The expression of each of RPM2, ARUKA, HOPX, ATP8A1, DOCK9, COL4A3, Clorf116, TTC37, IFT57, HSD17B6, NKX2-1 and MBIP may be assessed.

Obtaining expression information may comprise assessing protein expression, such as by ELISA, RIA, immunohistochemistry, or mass spectrometry. Alternatively, obtaining expression information expression comprises assessing mRNA expression, such as by quantitative RT-PCR, gene chip array expression, nCounter gene expression platform, and/or Northern blotting. The expression observed in said average adenocarcinoma of the lung may be relative to a pre-determined standard. The method may further comprising obtaining a biopsy from said patient, from which said cancer sample is obtained. The method may further comprise resecting said tumor. The adenocarcinoma of the lung may be early stage adenocarcinoma.

In another embodiment, there is provided a method of predicting the chemotherapeutic response of a human subject diagnosed with a non-small cell lung cancer tumor comprising obtaining expression information for 6 or more of the following genes in a non-small cell lung cancer sample obtained from said subject:
RPM2, ARUKA, HOPX, ATP8A1, DOCK9, COL4A3, Clorf116, TTC37, IFT57, HSD17B6, NKX2-1 and/or MBIP,
   wherein an alteration in the expression 6 or more of said genes, as compared to the average expression in non-small cell lung cancer, indicates that said subject will respond favorably to adjuvant chemotherapy. The decrease and/or increase of expression may be at least 0.2-fold. The method may further comprise treating said patient with adjuvant chemotherapy if predicted to be a responder. The adjuvant therapy comprises chemotherapy, such a platin compound and/or a taxane compound. The method may further comprise treating said subject with a non-chemotherapy cancer treatment if predicted to be a non-responder, such as radiotherapy. The method may further comprise obtaining expression information for 7, 8, 9. 10, 11, or all 12 of said genes.

Obtaining expression information may comprise assessing protein expression, such as by ELISA, RIA, immunohistochemistry, or mass spectrometry. Alternatively, obtaining expression information expression comprises assessing mRNA expression, such as by quantitative RT-PCR, gene chip array expression, nCounter gene expression platform, and/or Northern blotting. The expression observed in said average carcinoma of the lung, including adenocarcinoma, may be relative to a pre-determined standard. The method may further comprise obtaining a biopsy from said patient, from which said cancer sample is obtained. The method may further comprise resecting said tumor. The subject may have a stage I-III carcinoma of the lung, including adenocarcinomo.

The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the standard deviation of error for the device or method being employed to determine the value. Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention, which permit better understanding of the subject matter in combination with the detailed description of embodiments presented herein.

The patent or application file contains at least one drawing executed in color. Copies of this patent or patent application publication with color drawing(s) will be provided by the Office upon request and payment of the necessary fee.

FIGS. 1A-B. Schematic of the study design for: (FIG. 1A) the development and validation of the 18-hub-gene prognosis signature; and (FIG. 1B) the development and validation of the 12-gene predictive signature.

FIGS. 2A-B. (FIG. 2A) The topology of the constructed survival related gene network in NSCLC. The gene expression of 797 survival-related genes (false discovery rate<10%) from 442 ADC samples in the Consortium dataset was used to construct the gene network based on the Sparse PArtial Correlation Estimation (SPACE) algorithm. Each node represents one gene (only nodes with at least one connection are shown). The genes with at least 7 connections with other genes were identified as hub genes and labeled in red. (FIG. 2B) Composition of the 18 survival related hub genes. Network Connectivity refers to the number of genes that the hub gene has direct connection with based on the constructed gene network. The hazard ratios (HR) and P-values for each gene were derived from Cox models adjusted for age, cancer stage, and sample processing sites. P-values for synthetic lethal were from our previous study (Whitehurst et al., 2007), and P-values less than 0.05 were highlighted in yellow. Genetic alteration information was from the Tumorscape program (broadinstitute.org/tumorscape): “+” indicates the genes with significant amplification and “−” indicates significant deletion in lung cancer. The gene symbols of the 12 genes with either synthetic lethal or genetic alteration were highlighted in red.

FIGS. 3A-C. Validation of the 18-hub-gene signature in six independent data sets. (FIG. 3A) Lung ADC patients. (FIG. 3B) Stage I Lung ADC patients. (FIG. 3C) Lung SCC patients. The high- and low-risk groups were defined based on the 18-hub-gene signature which was derived from the Consortium data. The median of the estimated risk scores was used as the cut off to partition the patients into high-risk and low-risk groups. Red and black lines indicate predicted high- and low-risk groups. Red and black filled circles represent censored samples. Hazard ratio (HR) compares the overall survival of the high-risk group and the low-risk group. P values were obtained by the log-rank test. The patients with chemotherapy were excluded from the validation sets.

FIGS. 4A-D. Comparison of the 18-hub-gene set, 18-top-ranked-gene set and 797-SR-gene signature: (FIG. 4A) Summary of the prognostic performance for 18 hub-gene set, 18 top-ranked-gene set and 797-SR-gene set. The training data is the Consortium data; the validation sets include five different datasets. *Overall HR and p-values were calculated from meta-analysis. (FIG. 4B) Expression variation across the population in the Consortium dataset based on principal component analysis. (FIG. 4C) Pair-wise mutual information distance based on expression values in the Consortium dataset. (FIG. 4D) Entropy of expression values in the Consortium dataset.

FIGS. 5A-B. Validation of the 12-gene predictive signature in two independent data sets. (FIG. 5A) JBR.10 clinical trial dataset. The high- and low-risk groups were defined by the 12-gene signature. The patients were divided into two equal-sized risk groups based on their estimated risk scores. In the high-risk group, patients with ACT (pink line) have significantly longer survival time than patients without ACT (Observation group, blue line). In the low-risk group, patients with ACT (pink line) do not have significantly longer survival time than patients without ACT (Observation group, blue line). (FIG. 5B) UT Lung SPORE dataset. The risk groups were defined by the same 12-gene signature. In the high-risk group, patients with ACT (pink line) have significantly longer survival time than patients without ACT (Observation group, blue line). In the low-risk group, patients with ACT (pink line) do not have significantly longer survival time than patients without ACT.

FIGS. 6A-B. Different expression profiles of hub genes between the lung adenocarcinoma (ADC) group and lung squamous cell carcinoma (SCC) group. (FIG. 6A). Unsupervised clustering analysis of the microarray data from UT Lung SPORE data with 152 ADC patients and 57 SCC patients. Clustering was based on Ward's linkage criteria. The ADC patients are indicated with black color above the heat map. (FIG. 6B) P values were determined by two-sided student-t test comparing expression levels between two cancer histology subtypes.

FIG. 7A-B. Summary of the prognostic performance for the 12 hub-gene set.

### DESCRIPTION OF ILLUSTRATIVE EMBODIMENTS

In this study, the inventors used a systems biology approach to construct a survival-related gene network in NSCLC and identified 18 “hub” genes, which consistently co-expressed with many survival-related genes and hence play important roles in multiple biological processes. They showed that the 18-hub-gene set is functionally important and predicts the overall prognosis of NSCLC patients with Stage I-III disease. Previous RNAi screening study (Whitehurst et al., 2007) identified “synthetic lethal” genes; knockdown of these genes enhanced the cancer-killing effects of paclitaxel, which implies that these genes modulate chemotherapy drugs' effects in cancer cells. Recently, genetic aberration data have been successfully used to identify several key lung cancer driver genes in tumorigenesis (Weir et al., 2007). By integrating synthetic lethal genes and genetic aberration information with the hub genes, the inventors identified a 12-gene set that predicts ACT benefits in patients with Stage I-IIIA NSCLC. This 12-gene set was validated in two independent datasets, including the University of Texas Lung Specialized Program of Research Excellence (UT Lung SPORE) cohort (n=176) and the National Cancer Institute of Canada Clinical Trials Group JBR.10 clinical trial cohort (n=90). These and other aspects of the invention are described in detail below.

### I. LUNG CANCER

A. Lung Cancer

Lung cancer is a disease characterized by uncontrolled cell growth in tissues of the lung. If left untreated, this growth can spread beyond the lung in a process called metastasis into nearby tissue or other parts of the body. Most cancers that start in lung, known as primary lung cancers, are carcinomas that derive from epithelial cells. The main types of lung cancer are small-cell lung carcinoma (SCLC), also called oat cell cancer, and non-small-cell lung carcinoma (NSCLC). The most common symptoms are coughing (including coughing up blood), weight loss and shortness of breath.

The most common cause of lung cancer is long-term exposure to tobacco smoke, which causes 80-90% of lung cancers. Nonsmokers account for 10-15% of lung cancer cases, and these cases are often attributed to a combination of genetic factors, radon gas, asbestos, and air pollution including second-hand smoke. Lung cancer may be seen on chest radiograph and computed tomography (CT scan). The diagnosis is confirmed with a biopsy which is usually performed by bronchoscopy or CT-guidance. Treatment and long-term outcomes depend on the type of cancer, the stage (degree of spread), and the person's overall health, measured by performance status.

Common treatments include surgery, chemotherapy, and radiotherapy. NSCLC is sometimes treated with surgery, whereas SCLC usually responds better to chemotherapy and radiotherapy. Overall, 15% of people in the United States diagnosed with lung cancer survive five years after the diagnosis. Worldwide, lung cancer is the most common cause of cancer-related death in men and women, and is responsible for 1.38 million deaths annually, as of 2008.

Signs and symptoms that may suggest lung cancer include respiratory symptoms (coughing, coughing up blood, wheezing or shortness of breath), systemic symptoms (weight loss, fever, clubbing of the fingernails, or fatigue), and symptom due to local compress (chest pain, bone pain, superior vena cava obstruction, difficulty swallowing). If the cancer grows in the airway, it may obstruct airflow, causing breathing difficulties. The obstruction can lead to accumulation of secretions behind the blockage, and predispose to pneumonia.

Depending on the type of tumor, so-called paraneoplastic phenomena may initially attract attention to the disease. In lung cancer, these phenomena may include Lambert-Eaton myasthenic syndrome (muscle weakness due to autoantibodies), hypercalcemia, or syndrome of inappropriate antidiuretic hormone (SIADH). Tumors in the top of the lung, known as Pancoast tumors, may invade the local part of the sympathetic nervous system, leading to Horner's syndrome (dropping of the eyelid and a small pupil on that side), as well as damage to the brachial plexus.

Many of the symptoms of lung cancer (poor appetite, weight loss, fever, fatigue) are not specific. In many people, the cancer has already spread beyond the original site by the time they have symptoms and seek medical attention. Common sites of spread include the brain, bone, adrenal glands, opposite lung, liver, pericardium, and kidneys. About 10% of people with lung cancer do not have symptoms at diagnosis; these cancers are incidentally found on routine chest radiography.

Similar to many other cancers, lung cancer is initiated by activation of oncogenes or inactivation of tumor suppressor genes. Oncogenes are believed to make people more susceptible to cancer. Proto-oncogenes are believed to turn into oncogenes when exposed to particular carcinogens. Mutations in the K-ras proto-oncogene are responsible for 10-30% of lung adenocarcinomas. The epidermal growth factor receptor (EGFR) regulates cell proliferation, apoptosis, angiogenesis, and tumor invasion. Mutations and amplification of EGFR are common in non-small-cell lung cancer and provide the basis for treatment with EGFR-inhibitors. Her2/neu is affected less frequently. Chromosomal damage can lead to loss of heterozygosity. This can cause inactivation of tumor suppressor genes. Damage to chromosomes 3p, 5q, 13q, and 17p are particularly common in small-cell lung carcinoma. The p53 tumor suppressor gene, located on chromosome 17p, is affected in 60-75% of cases. Other genes that are often mutated or amplified are c-MET, NKX2-1, LKB1, PIK3CA, and BRAF.

Performing a chest radiograph is one of the first investigative steps if a person reports symptoms that may suggest lung cancer. This may reveal an obvious mass, widening of the mediastinum (suggestive of spread to lymph nodes there), atelectasis (collapse), consolidation (pneumonia), or pleural effusion. CT imaging is typically used to provide more information about the type and extent of disease. Bronchoscopy or CT-guided biopsy is often used to sample the tumor for histopathology.

Lung cancer often appears as a solitary pulmonary nodule on a chest radiograph.

However, the differential diagnosis is wide. Many other diseases can also give this appearance, including tuberculosis, fungal infections, metastatic cancer, or organizing pneumonia. Less common causes of a solitary pulmonary nodule include hamartomas, bronchogenic cysts, adenomas, arteriovenous malformation, pulmonary sequestration, rheumatoid nodules, Wegener's granulomatosis, or lymphoma. Lung cancer can also be an incidental finding, as a solitary pulmonary nodule on a chest radiograph or CT scan done for an unrelated reason. The definitive diagnosis of lung cancer is based on histological examination of the suspicious tissue in the context of the clinical and radiological features.

The three main subtypes of NSCLC are adenocarcinoma, squamous-cell lung carcinoma, and large-cell lung carcinoma. Nearly 40% of lung cancers are adenocarcinoma, which usually originates in peripheral lung tissue. Most cases of adenocarcinoma are associated with smoking; however, among people who have smoked fewer than 100 cigarettes in their lifetimes (“never-smokers”), adenocarcinoma is the most common form of lung cancer. A subtype of adenocarcinoma, the bronchioloalveolar carcinoma, is more common in female never-smokers, and may have a better long term survival. Squamous-cell carcinoma accounts for about 30% of lung cancers. They typically occur close to large airways. A hollow cavity and associated cell death are commonly found at the center of the tumor. About 9% of lung cancers are large-cell carcinoma. These are so named because the cancer cells are large, with excess cytoplasm, large nuclei and conspicuous nucleoli.

Lung cancer staging is an assessment of the degree of spread of the cancer from its original source. It is one of the factors affecting the prognosis and potential treatment of lung cancer. The initial evaluation of non-small-cell lung cancer (NSCLC) staging uses the TNM classification. This is based on the size of the primary tumor, lymph node involvement, and distant metastasis. After this, using the TNM descriptors, a group is assigned, ranging from occult cancer, through stages 0, IA (one-A), IB, IIA, IIB, IIIA, IIIB and IV (four). This stage group assists with the choice of treatment and estimation of prognosis. Small-cell lung carcinoma (SCLC) has traditionally been classified as ‘limited stage’ (confined to one half of the chest and within the scope of a single tolerable radiotherapy field) or ‘extensive stage’ (more widespread disease). However, the TNM classification and grouping are useful in estimating prognosis.

For both NSCLC and SCLC, the two general types of staging evaluations are clinical staging and surgical staging. Clinical staging is performed prior to definitive surgery. It is based on the results of imaging studies (such as CT scans and PET scans) and biopsy results. Surgical staging is evaluated either during or after the operation, and is based on the combined results of surgical and clinical findings, including surgical sampling of thoracic lymph nodes.

B. Carcinoma and Adenocarcinoma

Lung cancers are classified according to histological type. This classification is important for determining management and predicting outcomes of the disease. The vast majority of lung cancers are carcinomas—malignancies that arise from epithelial cells. Lung carcinomas are categorized by the size and appearance of the malignant cells seen by a histopathologist under a microscope. The two broad classes are non-small-cell and small-cell lung carcinoma. Adenocarcinoma of the lung is a common histological form of lung cancer that contains certain distinct malignant tissue architectural, cytological, or molecular features, including gland and/or duct formation and/or production of significant amounts of mucus.

Lung cancer is an extremely heterogeneous family of malignant neoplasms, with over 50 different histological variants recognized in the 4th revision of the World Health Organization (WHO) typing system, currently the most widely used lung cancer classification scheme. Because these variants have differing genetic, biological, and clinical properties, including response to treatment, correct classification of lung cancer cases are necessary to assure that lung cancer patients receive optimum management. While a small percentage of lung cancers are mainly sarcoma or tumors of hematopoietic or germ cell origin, approximately 98% of lung cancers are carcinoma, which are tumors composed of cells with epithelial characteristics. Adenocarcinomas (AdC's) are one of 8 major groups of lung carcinomas recognized in WHO-2004:
Squamous Cell Carcinoma
Small Cell Carcinoma
Adenocarcinoma
Large Cell Carcinoma
Adenosquamous Carcinoma
Sarcomatoid Carcinoma
Carcinoid Tumor
Salivary Gland-like Carcinoma

Adenocarcinoma is the most common type of lung cancer in lifelong non-smokers. Its incidence has been increasing in many developed Western nations in the past few decades, where it has become the most common major type of lung cancer in smokers (replacing squamous cell lung carcinoma) and in lifelong nonsmokers. According to the Nurses' Health Study, the risk of adenocarcinoma of the lung increases substantially after a long duration of previous tobacco smoking, with a previous smoking duration of 30 to 40 years giving a relative risk of approximately 2.4 compared to never-smokers, and a duration of more than 40 years giving a relative risk of approximately 5. Adenocarcinomas account for approximately 40% of lung cancers.

This cancer usually is seen peripherally in the lungs, as opposed to small cell lung cancer and squamous cell lung cancer, which both tend to be more centrally located, although it may also occur as central lesions. By unknown reasons, it often arises in relation to peripheral lung scars. The current theory is that the scar probably occurred secondary to the tumor, rather than causing the tumor. The adenocarcinoma has an increased incidence in smokers, and is the most common type of lung cancer seen in non-smokers and women. The peripheral location of adenocarcinoma in the lungs is due to the use of filters in cigarettes which prevent the larger particles from entering the lung. Generally, adenocarcinomas grow more slowly and form smaller masses than the other subtypes. However, they tend to form metastases widely at an early stage. Adenocarcinoma is a non-small cell lung carcinoma, and as such, it is not as responsive to radiation therapy as is small cell lung carcinoma, but is rather treated surgically, for example by pneumonectomy or lobectomy.

Adenocarcinomas are highly heterogeneous tumors, and several major histological subtypes are currently recognized: Acinar adenocarcinoma, Papillary adenocarcinoma, Bronchioloalveolar adenocarcinoma, and Solid adenocarcinoma with mucin production. In as many as 80% of tumors that are extensively sampled, components of more than one of these subtypes will be recognized. In such cases, the tumors should be classified as a fifth “subtype,” namely “adenocarcinoma, mixed subtypes.”

Adenocarcinoma of the lung tends to stain mucin positive as it is derived from the mucus producing glands of the lungs. Similar to other adenocarcinoma, if this tumor is well differentiated (low grade) it will resemble the normal glandular structure. Poorly differentiated adenocarcinoma will not resemble the normal glands (high grade) and will be detected by seeing that they stain positive for mucin (which the glands produce).

To reveal the adenocarcinomatous lineage of the solid variant, demonstration of intracellular mucin production may be performed. Foci of squamous metaplasia and dysplasia may be present in the epithelium proximal to adenocarcinomas, but these are not the precursor lesions for this tumor. Rather, the precursor of peripheral adenocarcinomas has been termed atypical adenomatous hyperplasia (AAH). Microscopically, AAH is a well-demarcated focus of epithelial proliferation, containing cuboidal to low-columnar cells resembling Clara cells or type II pneumocytes. These demonstrate various degrees of cytologic atypia, including hyperchromasia, pleomorphism, prominent nucleoli. However, the atypia is not to the extent as seen in frank adenocarcinomas. Lesions of AAH are monoclonal, and they share many of the molecular aberrations (like KRAS mutations) that are associated with adenocarcinomas.

C. Therapy

Treatment for lung cancer depends on the cancer's specific cell type, how far it has spread, and the person's performance status. Common treatments include palliative care, surgery, chemotherapy, and radiation therapy.

If investigations confirm NSCLC, the stage is assessed to determine whether the disease is localized and amenable to surgery or if it has spread to the point where it cannot be cured surgically. CT scan and positron emission tomography are used for this determination. If mediastinal lymph node involvement is suspected, mediastinoscopy may be used to sample the nodes and assist staging. Blood tests and pulmonary function testing are used to assess whether a person is well enough for surgery. If pulmonary function tests reveal poor respiratory reserve, surgery may not be a possibility.

In most cases of early-stage NSCLC, removal of a lobe of lung (lobectomy) is the surgical treatment of choice. In people who are unfit for a full lobectomy, a smaller sublobar excision (wedge resection) may be performed. However, wedge resection has a higher risk of recurrence than lobectomy. Radioactive iodine brachytherapy at the margins of wedge excision may reduce the risk of recurrence. Rarely, removal of a whole lung (pneumonectomy) is performed. Video-assisted thoracoscopic surgery and VATS lobectomy use a minimally invasive approach to lung cancer surgery. VATS lobectomy is equally effective compared to conventional open lobectomy, with less postoperative illness.

Radiotherapy is often given together with chemotherapy, and may be used with curative intent in people with NSCLC who are not eligible for surgery. This form of high-intensity radiotherapy is called radical radiotherapy. A refinement of this technique is continuous hyperfractionated accelerated radiotherapy (CHART), in which a high dose of radiotherapy is given in a short time period. Postoperative thoracic radiotherapy generally should not be used after curative intent surgery for NSCLC. Some people with mediastinal N2 lymph node involvement might benefit from post-operative radiotherapy.

If cancer growth blocks a short section of bronchus, brachytherapy (localized radiotherapy) may be given directly inside the airway to open the passage. Compared to external beam radiotherapy, brachytherapy allows a reduction in treatment time and reduced radiation exposure to healthcare staff.

Recent improvements in targeting and imaging have led to the development of stereotactic radiation in the treatment of early-stage lung cancer. In this form of radiotherapy, high doses are delivered in a small number of sessions using stereotactic targeting techniques. Its use is primarily in patients who are not surgical candidates due to medical comorbidities.

For both NSCLC and SCLC patients, smaller doses of radiation to the chest may be used for symptom control (palliative radiotherapy).

The chemotherapy regimen depends on the tumor type. Small-cell lung carcinoma (SCLC), even relatively early stage disease, is treated primarily with chemotherapy and radiation. In SCLC, cisplatin and etoposide are most commonly used. Combinations with carboplatin, gemcitabine, paclitaxel, vinorelbine, topotecan, and irinotecan are also used. In advanced non-small cell lung carcinoma (NSCLC), chemotherapy improves survival and is used as first-line treatment, provided the person is well enough for the treatment. Typically, two drugs are used, of which one is often platinum-based (either cisplatin or carboplatin). Other commonly used drugs are gemcitabine, paclitaxel, docetaxel, pemetrexed, etoposide or vinorelbine.

Adjuvant chemotherapy refers to the use of chemotherapy after apparently curative surgery to improve the outcome. In NSCLC, samples are taken of nearby lymph nodes during surgery to assist staging. If stage II or III disease is confirmed, adjuvant chemotherapy improves survival by 5% at five years. The combination of vinorelbine and cisplatin is more effective than older regimens. Adjuvant chemotherapy for people with stage 1B cancer is controversial, as clinical trials have not clearly demonstrated a survival benefit. Trials of preoperative chemotherapy (neoadjuvant chemotherapy) in resectable NSCLC have been inconclusive.

In people with terminal disease, palliative care or hospice management may be appropriate. These approaches allow additional discussion of treatment options and provide opportunities to arrive at well-considered decisions and may avoid unhelpful but expensive care at the end of life.

Chemotherapy may be combined with palliative care in the treatment of the NSCLC. In advanced cases, appropriate chemotherapy improves average survival over supportive care alone, as well as improving quality of life. With adequate physical fitness, maintaining chemotherapy during lung cancer palliation offers 1.5 to 3 months of prolongation of survival, symptomatic relief, and an improvement in quality of life, with better results seen with modern agents. The NSCLC Meta-Analyses Collaborative Group recommends if the recipient wants and can tolerate treatment, then chemotherapy should be considered in advanced NSCLC.

### II. GENE SIGNATURES

In accordance with the methods described in greater detail in the following Examples, the inventors have identified an 18-gene signature for predicting prognosis, and a 12-gene signature for predicting response to adjuvant chemotherapy. The expression of gene products listed below will be instructive in prognosis and predicting response:
18-Gene Signature
   RPM2
   ARUKA
   CDKN3
   PRC1
   HOPX
   DPP4
   ATP8A1
   CYP2B6
   DOCK9
   COL4A3
   Clorf116
   TTC37
   IFT57
   HSD17B6
   NKX2-1
   GPR116
   MBIP
   SLC35A5
      Up-regulated genes in the high risk group include RPM2, ARUKA, CDKN3, PRC1, DOCK9, TTC37 and SLC35A5, while up-regulated genes in the low risk group include HOPX, DPP4, ATP8A1, CYP2B6, COL4A3, Clorf116, IFT57, HSD17B6, NKX2-1, GPR116, and MBIP.
12-Gene Signature
   RPM2
   ARUKA
   HOPX
   ATP8A1
   DOCK9
   COL4A3
   Clorf116
   TTC37
   IFT57
   HSD17B6
   NKX2-1
   MBIP
      Up-regulated genes in the responders include RPM2, ARUKA, DOCK9, and TTC37, and up-regulated genes in the non-responders include HOPX, ATP8A1, COL4A3, Clorf116, IFT57, HSD17B6, NKX2-1, and MBIP.

The gene expression profile for patient samples can be prepared using a variety of different methods. The profile may be evaluated for the presence of one or more of the gene signatures by scoring or classifying the patient profile against each gene signature. Various classification schemes are known for classifying samples between two or more classes or groups, and these include, without limitation: Principal Components Analysis, Naïve Bayes, Support Vector Machines, Nearest Neighbors, Decision Trees, Logistic, Artificial Neural Networks, and Rule-based schemes. In addition, the predictions from multiple models can be combined to generate an overall prediction. For example, a “majority rules” prediction may be generated from the outputs of a Naïve Bayes model, a Support Vector Machine model, and a Nearest Neighbor model. The classifier algorithm may be supervised or semi-supervised in some embodiments.

Thus, a classification algorithm or “class predictor” may be constructed to classify samples. The process for preparing a suitable class predictor is reviewed in R. Simon, (2003), which review is hereby incorporated by reference in its entirety.

Generally, the gene expression profiles for patient specimens are scored or classified as high risk or low risk, including with stratified or continuous intermediate classifications or scores reflective of risk of recurrence or other event. As discussed, such signatures may be assembled from gene expression data disclosed herein, or prepared from independent data sets. The signatures may be stored in a database and correlated to patient tumor gene expression profiles in response to user inputs.

After comparing the patient's gene expression profile to the signature, the sample is classified as, or for example, given a probability of being, a high risk profile or a low risk profile. The classification may be determined computationally based upon known methods as described above. The result of the computation may be displayed on a computer screen or presented in a tangible form, for example, as a probability (e.g., from 0 to 100%) of the patient having a given survival period, or their ability to respond to a course of treatment, such as selection of adjuvant chemotherapy.

Furthermore, it is within the general scope of the present invention to provide methods for the detection of mRNA and proteins from the list above. Any method of detection known to one of skill in the art falls within the general scope of the present invention.

A. Nucleic Acid Detection

Nucleic acid sequences disclosed herein will find use in detecting expression of target genes, e.g., as probes or primers for embodiments involving nucleic acid hybridization. As used in this application, the term “polynucleotide” refers to a nucleic acid molecule that has been isolated essentially or substantially free of total genomic nucleic acid to permit hybridization and amplification, but is not limited to such. An oligonucleotide refers to a nucleic acid molecule that is complementary or identical to at least 5 contiguous nucleotides of a given sequence.

It also is contemplated that a particular polypeptide from a given species may be represented by natural variants that have slightly different nucleic acid sequences but, nonetheless, encode the same protein. In this respect, the term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants.

A nucleic acid may be of the following lengths: about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs.

1. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772 and U.S. Patent Publication 2008/0009439. The relevant portions of these and other references identified in this section of the Specification are incorporated herein by reference.

2. In situ Hybrization

In situ hybridization (ISH) is a type of hybridization that uses a labeled complementary DNA or RNA strand (i.e., probe) to localize a specific DNA or RNA sequence in a portion or section of tissue (in situ), or, if the tissue is small enough (e.g. plant seeds, Drosophila embryos), in the entire tissue (whole mount ISH). This is distinct from immunohistochemistry, which localizes proteins in tissue sections. Fluorescent DNA ISH (FISH) can, for example, be used in medical diagnostics to assess chromosomal integrity. RNA ISH (hybridization histochemistry) is used to measure and localize mRNAs and other transcripts within tissue sections or whole mounts.

For hybridization histochemistry, sample cells and tissues are usually treated to fix the target transcripts in place and to increase access of the probe. As noted above, the probe is either a labeled complementary DNA or, now most commonly, a complementary RNA (riboprobe). The probe hybridizes to the target sequence at elevated temperature, and then the excess probe is washed away (after prior hydrolysis using RNase in the case of unhybridized, excess RNA probe). Solution parameters such as temperature, salt and/or detergent concentration can be manipulated to remove any non-identical interactions (i.e., only exact sequence matches will remain bound). Then, the probe that was labeled with either radio-, fluorescent- or antigen-labeled bases (e.g., digoxigenin) is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

3. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to any sequence corresponding to a nucleic acid sequence are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 2001). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Reverse transcription (RT) of RNA to cDNA followed by quantitative PCR (RT-PCR) can be used to determine the relative concentrations of specific mRNA species isolated from a cell. By determining that the concentration of a specific mRNA species varies, it is shown that the gene encoding the specific mRNA species is differentially expressed. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR products must be sampled when the PCR reactions are in the linear portion of their curves.

A second condition for an RT-PCR experiment is to determine the relative abundances of a particular mRNA species. Typically, relative concentrations of the amplifiable cDNAs are normalized to some independent standard. The goal of an RT-PCR experiment is to determine the abundance of a particular mRNA species relative to the average abundance of all mRNA species in the sample.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

RT-PCR can be performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Various nucleic acid detection methods known in the art are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Chip Technologies

Specifically contemplated by the present inventor are chip-based DNA technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of a gene target.

5. Nucleic Acid Arrays

The present invention may involve the use of arrays or data generated from an array. Data may be readily available. An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g., up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon Such arrays may vary in a number of different ways, including average probe length, sequence or types of probes, nature of bond between the probe and the array surface, e.g., covalent or non-covalent, and the like. The labeling and screening methods of the present invention and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610;287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

B. Protein Detection

In certain embodiments, the present invention concerns determining the expression level of a protein corresponding to a target gene. In certain embodiments, the proteinaceous composition may be identified using an antibody. In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (e.g., alter pH, ionic strength, and temperature).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that by itself does not adsorb molecules to any significant extent and that has a broad range of chemical, physical and thermal stability. The ligand should be coupled in such a way as to not affect its binding properties. The ligand also should provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

In some embodiments, the present invention concerns immunodetection methods. Immunodetection methods include enzyme linked immunosorbent assay (ELISA), radioimmunoassay (RIA), immunoradiometric assay, fluoroimmunoassay, chemiluminescent assay, bioluminescent assay, and Western blot, though several others are well known to those of ordinary skill. The steps of various useful immunodetection methods have been described in the scientific literature, such as, e.g., Doolittle et al. (1999); Gulbis et al. (1993); De Jager et al. (1993); and Nakamura et al. (1987), each incorporated herein by reference.

In general, the immunobinding methods include obtaining a sample containing a protein, polypeptide and/or peptide, and contacting the sample with a first antibody, monoclonal or polyclonal, in accordance with the present invention, as the case may be, under conditions effective to allow the formation of immunocomplexes.

These methods include methods for purifying a protein, polypeptide and/or peptide from organelle, cell, tissue or organism's samples. In these instances, the antibody removes the antigenic protein, polypeptide and/or peptide component from a sample. The antibody will preferably be linked to a solid support, such as in the form of a column matrix, and the sample suspected of containing the protein, polypeptide and/or peptide antigenic component will be applied to the immobilized antibody. The unwanted components will be washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen or antigenic domain, and contact the sample with an antibody against the antigen or antigenic domain, and then detect and quantify the amount of immune complexes formed under the specific conditions.

In terms of antigen detection, the biological sample analyzed may be any sample that is suspected of containing an antigen or antigenic domain, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

1. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, antibodies are immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and/or then contacted with antibodies. After binding and/or washing to remove non-specifically bound immune complexes, the bound anti-antibodies are detected. Where the initial antibodies are linked to a detectable label, the immune complexes may be detected directly. Again, the immune complexes may be detected using a second antibody that has binding affinity for the first antibody, with the second antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. An example of a washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. This may be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

2. Immunohistochemistry

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Immunohistochemistry or IHC refers to the process of localizing proteins in cells of a tissue section exploiting the principle of antibodies binding specifically to antigens in biological tissues. It takes its name from the roots “immuno,” in reference to antibodies used in the procedure, and “histo,” meaning tissue. Immunohistochemical staining is widely used in the diagnosis and treatment of cancer.

Visualising an antibody-antigen interaction can be accomplished in a number of ways. In the most common instance, an antibody is conjugated to an enzyme, such as peroxidase, that can catalyse a colour-producing reaction. Alternatively, the antibody can also be tagged to a fluorophore, such as FITC, rhodamine, Texas Red, Alexa Fluor, or DyLight Fluor. The latter method is of great use in confocal laser scanning microscopy, which is highly sensitive and can also be used to visualize interactions between multiple proteins.

Briefly, frozen-sections may be prepared by rehydrating 50 mg of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

There are two strategies used for the immunohistochemical detection of antigens in tissue, the direct method and the indirect method. In both cases, the tissue is treated to rupture the membranes, usually by using a kind of detergent called Triton X-100.

The direct method is a one-step staining method, and involves a labeled antibody (e.g. FITC conjugated antiserum) reacting directly with the antigen in tissue sections. This technique utilizes only one antibody and the procedure is therefore simple and rapid. However, it can suffer problems with sensitivity due to little signal amplification and is in less common use than indirect methods.

The indirect method involves an unlabeled primary antibody (first layer) which reacts with tissue antigen, and a labeled secondary antibody (second layer) which reacts with the primary antibody. The secondary antibody must be against the IgG of the animal species in which the primary antibody has been raised. This method is more sensitive due to signal amplification through several secondary antibody reactions with different antigenic sites on the primary antibody. The second layer antibody can be labeled with a fluorescent dye or an enzyme.

In a common procedure, a biotinylated secondary antibody is coupled with streptavidin-horseradish peroxidase. This is reacted with 3,3′-Diaminobenzidine (DAB) to produce a brown staining wherever primary and secondary antibodies are attached in a process known as DAB staining. The reaction can be enhanced using nickel, producing a deep purple/gray staining.

The indirect method, aside from its greater sensitivity, also has the advantage that only a relatively small number of standard conjugated (labeled) secondary antibodies needs to be generated. For example, a labeled secondary antibody raised against rabbit IgG, which can be purchased “off the shelf,” is useful with any primary antibody raised in rabbit. With the direct method, it would be necessary to make custom labeled antibodies against every antigen of interest.

3. Protein Arrays

Protein array technology is discussed in detail in Pandey and Mann (2000) and MacBeath and Schreiber (2000), each of which is herein specifically incorporated by reference.

These arrays, typically contain thousands of different proteins or antibodies spotted onto glass slides or immobilized in tiny wells, allow one to examine the biochemical activities and binding profiles of a large number of proteins at once. To examine protein interactions with such an array, a labeled protein is incubated with each of the target proteins immobilized on the slide, and then one determines which of the many proteins the labeled molecule binds. In certain embodiments such technology can be used to quantitate a number of proteins in a sample.

The basic construction of protein chips has some similarities to DNA chips, such as the use of a glass or plastic surface dotted with an array of molecules. These molecules can be DNA or antibodies that are designed to capture proteins. Defined quantities of proteins are immobilized on each spot, while retaining some activity of the protein. With fluorescent markers or other methods of detection revealing the spots that have captured these proteins, protein microarrays are being used as powerful tools in high-throughput proteomics and drug discovery.

The earliest and best-known protein chip is the ProteinChip by Ciphergen Biosystems Inc. (Fremont, Calif.). The ProteinChip is based on the surface-enhanced laser desorption and ionization (SELDI) process. Known proteins are analyzed using functional assays that are on the chip. For example, chip surfaces can contain enzymes, receptor proteins, or antibodies that enable researchers to conduct protein-protein interaction studies, ligand binding studies, or immunoassays. With state-of-the-art ion optic and laser optic technologies, the ProteinChip system detects proteins ranging from small peptides of less than 1000 Da up to proteins of 300 kDa and calculates the mass based on time-of-flight (TOF).

The ProteinChip biomarker system is the first protein biochip-based system that enables biomarker pattern recognition analysis to be done. This system allows researchers to address important clinical questions by investigating the proteome from a range of crude clinical samples (i.e., laser capture microdissected cells, biopsies, tissue, urine, and serum). The system also utilizes biomarker pattern software that automates pattern recognition-based statistical analysis methods to correlate protein expression patterns from clinical samples with disease phenotypes.

### III. TREATMENT OF CANCER

In some embodiments, the invention further provides treatment of cancer—in particular, lung cancer. One of skill in the art will be aware of many treatments and treatment combinations may be used, some but not all of which are described below.

A. Formulations and Routes for Administration to Patients

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions. Of particular interest is direct intratumoral administration, perfusion of a tumor, or administration local or regional to a tumor, for example, in the local or regional vasculature or lymphatic system, or in a resected tumor bed (e.g., post-operative catheter). For practically any tumor, systemic delivery also is contemplated. This will prove especially important for attacking microscopic or metastatic cancer.

The active compounds may also be administered as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The actual dosage amount of a composition of the present invention administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

“Treatment” and “treating” refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

A “disease” can be any pathological condition of a body part, an organ, or a system resulting from any cause, such as infection, genetic defect, and/or environmental stress.

“Prevention” and “preventing” are used according to their ordinary and plain meaning to mean “acting before” or such an act. In the context of a particular disease, those terms refer to administration or application of an agent, drug, or remedy to a subject or performance of a procedure or modality on a subject for the purpose of blocking the onset of a disease or health-related condition.

The subject can be a subject who is known or suspected of being free of a particular disease or health-related condition at the time the relevant preventive agent is administered. The subject, for example, can be a subject with no known disease or health-related condition (i.e., a healthy subject).

In additional embodiments of the invention, methods include identifying a patient in need of treatment. A patient may be identified, for example, based on taking a patient history or based on findings on clinical examination.

B. Cancer Treatments

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein transferase inhibitors, transplatinum, 5-fluorouracil, vincristin, vinblastin and methotrexate and pharmaceutically acceptable salts, acids or derivatives of any of the above.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants, e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Gene Therapy

In yet another embodiment, the gene therapy may be applied to the subject. Suitable genes included inducers of cellular proliferation, tumor suppressors, or regulators of programmed cell death.

6. Other Agents

It is contemplated that other agents may be used with the present invention. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

C. Dosage

The amount of therapeutic agent to be included in the compositions or applied in the methods set forth herein will be whatever amount is pharmaceutically effective and will depend upon a number of factors, including the identity and potency of the chosen therapeutic agent. One of ordinary skill in the art would be familiar with factors that are involved in determining a therapeutically effective dose of a particular agent. Thus, in this regards, the concentration of the therapeutic agent in the compositions set forth herein can be any concentration. In some particular embodiments, the total concentration of the drug is less than 10%. In more particular embodiments, the concentration of the drug is less than 5%. The therapeutic agent may be applied once or more than once. In non-limiting examples, the therapeutic agent is applied once a day, twice a day, three times a day, four times a day, six times a day, every two hours when awake, every four hours, every other day, once a week, and so forth. Treatment may be continued for any duration of time as determined by those of ordinary skill in the art.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Patients and Samples.

UT Lung SPORE Cohort.

Patients were eligible to enter the study if they underwent curative resection for NSCLC at MD Anderson Cancer Center between December 1996 and June 2007. Those with radiation therapy were excluded from the study. All tissue samples were obtained by surgical resection from patients who had provided written informed consent. Tissues were stored at −140° C. after being snap frozen in liquid nitrogen. Serial sectioning of each sample was used to histologically evaluate tumor and malignant cells content before RNA extraction (Maitra et al., 2001). The primary tumor tissues from 176 patients were randomly selected from the UT Lung SPORE tumor collection based on stringent, predefined quality control procedures, including the presence of ≧70% tumor tissue and ≧50% malignant cells in the frozen tissue used for RNA extraction. In this cohort, 133 patients are adenocarcinomas (ADCs) and 43 patients are squamous cell carcinomas (SCCs); 49 patients received ACT (mainly carboplatin plus taxanes) and 127 patients did not receive ACT. The clinical information together with gene expression data for the UT Lung SPORE cohort were deposited in GEO database (GSE42127).

Samples from Other Groups.

In addition to the UT Lung SPORE data, 7 public NSCLC microarray datasets (Lee et al., 2008; Shedden et al., 2008; and Zhu et al., 2010; Bild et al., 2006; Matsuyama et al., 2011; Raponi et al., 2006 and Tomida et al., 2009) were used in this study. The National Cancer Institute Director's Challenge Consortium study (Consortium dataset) (Shedden et al., 2008), which is the largest independent public available lung cancer microarray dataset and involves 442 resected ADCs, was used as the training set. Six datasets were used to validate the prognosis signature: UT lung SPORE data, GSE3141 (ADC n=58, SCC n=53), GSE8894 (ADC n=62, SCC n=76), GSE11969 (ADC n=90, and SCC n=35), GSE13213 (ADC n=117), GSE4573 (SCC n=129). Among these 6 datasets, three (GSE13213, GSE8894 and GSE11969) are Asian cohorts. Two datasets were used to validate the predictive signature: UT lung SPORE data and GSE14814 that includes 90 samples (49 patients with vinorelbine plus cisplatin ACT and 41 patients without ACT) collected from the JBR.10 trial. Table 5 provides detailed information on these datasets. Since 43 out of 133 samples in the original JBR.10 dataset (GSE14814) were also included in the Consortium data (training set), these 43 samples were excluded from the JBR.10 dataset to ensure the independence between the training and validation sets.

TABLE 5
Clinical characteristics of patients in the validation datasets

| | SPORE | GSE13213 | GSE 11969 | GSE8894 | GSE3141 | GSE4573 | GSE14814 |
|---|---|---|---|---|---|---|---|
| | New data | Tomida2009 | Matsuyama2011 | Lee2008 | Bild2006 | Raponi2006 | Zhu2010 |
| Total Patients | n = 176 | n = 117 | n = 149 | n = 138 | n = 111 | n = 129 | n = 90 |
| Gender | | | | | | | |
| Female | 83 (47.2%) | 57 (48.7%) | 48 (32.2%) | 34 (24.6%) | — | 47 (36.4%) | 23 (25.6%) |
| Male | 93 (52.8%) | 60 (51.3%) | 101 (67.8%) | 104 (75.4%) | — | 82 (63.6%) | 67 (74.4%) |
| Stage | | | | | | | |
| I | 112 (63.6%) | 79 (67.5%) | 78 (52.3%) | — | 62 (55.9%) | 73 (56.6%) | 45 (50.0%) |
| II | 32 (18.2%) | 13 (11.1%) | 26 (17.4%) | — | — | 33 (25.6%) | 45 (50.0%) |
| III | 30 (17.0%) | 25 (21.4%) | 45 (30.2%) | — | — | 23 (17.8%) | — |
| IV | 1 (0.6%) | — | — | — | — | — | — |
| Unknown | 1 (0.6%) | — | — | 138 (100%) | 49 (44.1%) | — | — |
| Histology | | | | | | | |
| ADCs | 133 (75.6%) | 117 (100%) | 90 (60.4%) | 62 (44.9%) | 58 (52.3%) | — | 28 (31.1%) |
| SCCs | 43 (24.4%) | — | 35 (23.5%) | 76 (55.1%) | 53 (47.7%) | 129 (100%) | 52 (57.8%) |
| Others | — | — | 24 (16.1%) | — | — | — | 10 (11.1%) |
| Median Follow-up | 47.4 | 68 | 78 | 41.8 | 31.1 | 34.2 | 64.8 |
| (Months) | | | | | | | |
| Platform | Illumina | Agilent 44K | Agilent 21.6K | Affy U133 | Affy. U133 | Affy. U133A | Affy. U133A |
| | Human-WG6 | | custom array | Plus_2 | Plus_2 | | |
| | V3 | | | | | | |

RNA Extraction and Microarray Profiling.

The frozen tissues specimens were processed on the cryostat to generate multiple 5-micron thick sections for subsequent homogenization using an electric homogenizer. Before RNA extraction, histology sections were stained and reviewed to assess the percentage of tumor. Total RNA was extracted using TRIREAGENT (Life Technologies, NY, USA) according to manufacturer's protocol. The nanodrop spectrophotometer (Thermo Fisher, Wilmington, Del., USA) was used to estimate the concentration of RNA while the quality of the RNA was assessed on Nano Series II RNA LAB-chips using Agilent Bioanalyzer 2100 (Agilent Technologies, Inc., Santa Clara, Calif., USA). All samples selected for RNA profiling have an RNA integrity number (RIN)≧5. Total RNA was processed for analysis on the Illumina Human-6 V3 arrays according to Illumina protocols for first- and second-strand synthesis, biotin labeling and fragmentation.

Microarray Data Preprocessing.

The UT Lung SPORE Illumina beadarray data were processed using Model-Based Background Correction (MBCB) method (Xie et al., 2009). For the Consortium and GSE14814 datasets, the raw Affymetrix .cel data was downloaded from caArray database and Gene Expression Omnibus (GEO), respectively. Both datasets were then preprocessed by the Robust Multiarray Average (RMA) algorithm and quantile-quantile normalization (Irizarry et al., 2003). For datasets that did not provide raw data file (GSE3141, GSE4573, and GSE8894) or used the Agilent platforms (GSE11969 and GSE13213), the inventors downloaded the author processed data from GEO. All gene expression values were log 2 transformed. The EntrezlDs were used to map genes across microarray platforms.

Survival Analysis.

Overall survival time was calculated from the date of surgery until death or last follow-up contact. Survival curves were estimated using the Kaplan-Meier method (Kaplan and Meier, 1958) and were compared using log-rank test. Univariate and multivariate survival analyses were performed using Cox proportional-hazards model (Collett 2003). Meta-analysis was used to combine the results across different test sets. It was performed using the R package metagen (Schwarzer 2012). The overall combined estimate of the hazard ratio was estimated based on their values and standard errors in individual validation set.

Gene Network Analysis.

The lung cancer survival-related gene network was constructed using the Consortium dataset. The association between the expression level of each probeset and survival time was evaluated using multivariate Cox model adjusted for age, cancer stage, and sample processing sites. The false discovery rate (FDR) was calculated from a beta-uniform mixture model (Pounds S, Morris SW. Estimating the occurrence of false positives and false negatives in microarray studies by approximating and partitioning the empirical distribution of p-values. Bioinformatics 2003; 19:1236-42 35). All probesets that passed the FDR criteria (FDR<10%) were included in gene network analysis. When there are multiple probesets corresponding to a single gene, the expression levels from the probesets were averaged to derive the gene level expression. The Sparse PArtial Correlation Estimation (SPACE) algorithm (Peng et al., 2009) was used to construct the network of survival-associated genes using their expression values in the Consortium dataset. From the constructed gene network, genes with at least 7 connections to other genes were identified as “hub” genes.

Mapping Across Different Microarray Platforms.

To validate signatures across different studies, the inventors followed several steps for probe mapping across microarray platforms (Allen et al., 2012): (1) the inventors identified the gene symbol of each probe based on the annotation from the vendor or GEO; (2) gene-level expression values were derived by averaging all probes mapped to the same gene symbol; and (3) the gene expression values of the signature were extracted for testing. If the genes in the signatures cannot be mapped to a testing set, they set the expression values of the missing genes to a constant value, which is the median expression value of all available signature genes, to minimize influence from these missing genes.

Prediction Methods.

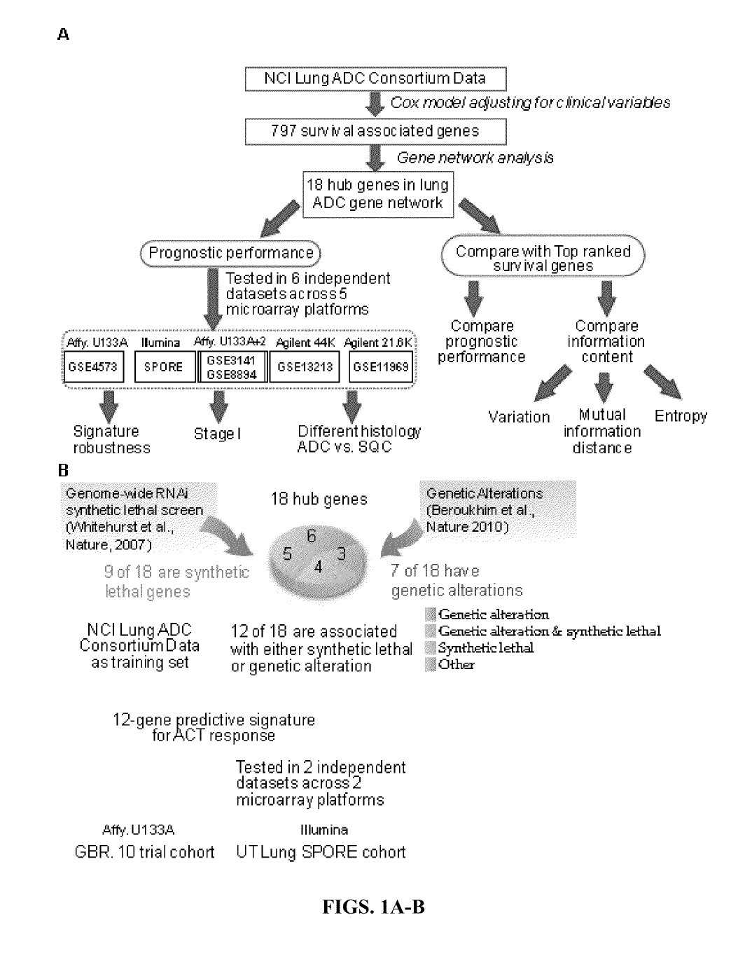

Supervised principal component analysis (Bair et al., 2004 and Breiman et al., 1984) was applied to construct the prediction model, which is based on the linear combinations of gene expressions of the provided gene set in the training data set. Then the inventors apply the risk prediction model to the test set, and derive a risk score for each samples based on their gene expressions. The test set samples are divided in to two equal-sized risk groups based on the median of the predicted risk scores. For the prediction model, they used the first 3 principal components, which the default parameter of the program with prediction (superPC R package). The training and validation strategy is illustrated in FIG. 1.

Analysis of Information Content.

The mutual information is a measure of the mutual dependence of two random variables. In this study, the mutual information distance, which quantifies the mutual information between the expression levels of two genes, is calculated using R package BioDist. Differential entropy measures information content for continuous random variables. It is defined by

h(X)=−∫_{X}ƒ(x)log ƒ(x)dx

where ƒ(x) is the probability density function of the random variable X. In this study, the entropy is calculated by assuming the gene expression values are from a multivariate Gaussian distribution.

### Example 2

### Results

Identification of an 18-Hub-Gene Set.

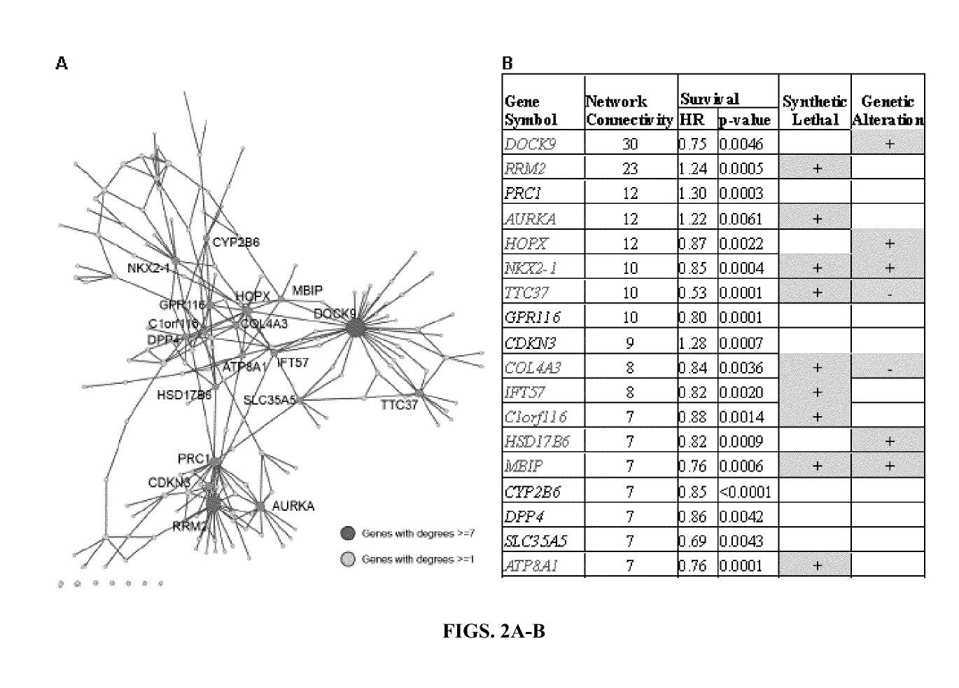

From the Consortium dataset, the inventors identified 797 genes (FIGS. 1A-B) whose expression levels were associated with patients' overall survival time (FDR <10%). Next, they constructed a lung cancer survival-related gene network (see Method Section) based on expression changes of these 797 genes across 442 lung cancer samples in the Consortium dataset (FIG. 2A). The inventors identified 18 hub genes that are connected with at least 7 other genes in the constructed network. Among these 18 genes (summarized in FIG. 2B), RRM2, AURKA, PRC1, and CDKN3 are associated with poor prognosis, while the remaining 14 genes are associated with good prognosis.

Prognosis Performance of the 18-Hub-Gene Set.

Robustness of the Prognostic Signature.

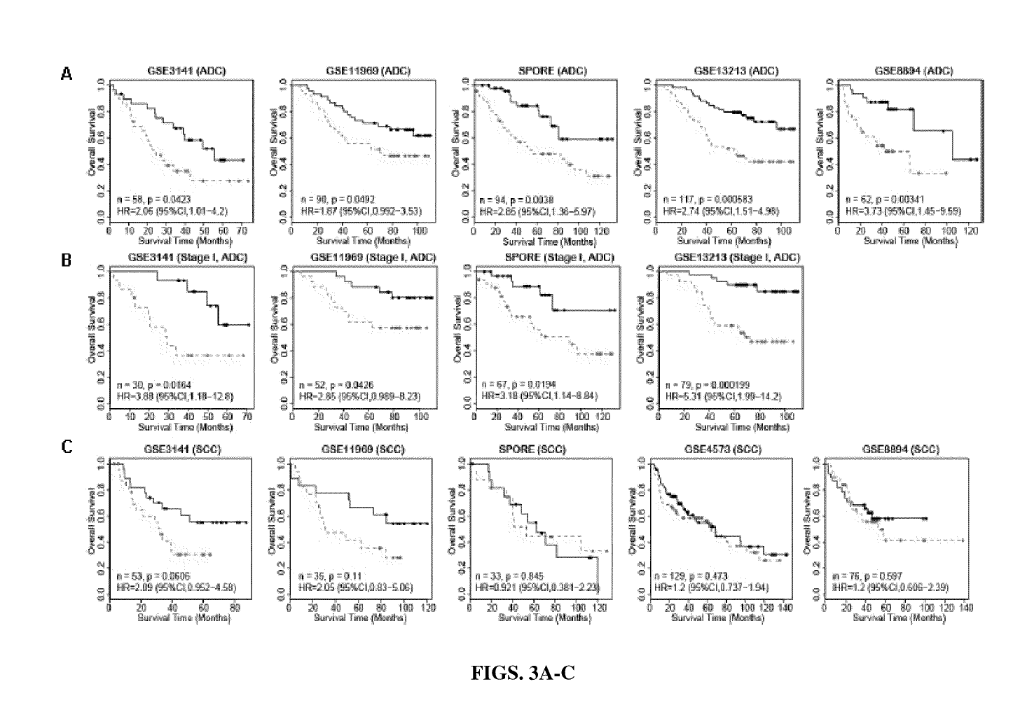

A prognostic signature was developed using the expression of the 18-hub-gene set and patients' survival outcomes from the Consortium dataset (training set) based on the superPC method. The prognostic signature was evaluated in ADC patients from 5 independent validation sets across 4 different microarray platforms, including: UT Lung SPORE (Illumina-6 V3), GSE3141 and GSE8894 (Affymetrix U133Plus2), GSE11969 (Agilent 21.6K custom array) and GSE13213 (Agilent 44K). Patients receiving adjuvant chemotherapy were excluded from the validation sets. Remarkably, the prognostic signature consistently predicted overall survival in all 5 validation sets. The predicted high-risk group has significantly worse survival outcomes than the predicted low-risk group: GSE3141 (n=58, HR=2.06 [1.01-4.2], p=0.042), UT Lung SPORE (n=94, HR=2.85 [1.36-5.97], p=0.0038), GSE8894 (n=62, HR=3.73 [1.45-9.59], p=0.0034), GSE11969 (n=90, HR=1.87 [0.99-3.53], p=0.049), GSE13213 (n=117, HR=2.74 [1.51-4.98], p=0.00058) (FIG. 3A). Since most of the public datasets did not provide complete demographic information, the inventors performed multivariate survival analysis in UT Lung SPORE data. The predicted high-risk group has significantly worse survival outcomes than the predicted low-risk group (HR=2.93 [1.25-6.88], p=0.0137) after adjusting for stage, age and gender (Table 2). Furthermore, the 18-hub-gene signature consistently predicted the prognosis of patients with stage I disease: GSE3141 (n=30, HR=3.88 [1.18-12.8], p=0.016), UT Lung SPORE (n=67, HR=3.18 [1.14-8.84], p=0.019), GSE11969 (n=52, HR=2.85 [0.99-8.23], p=0.043), GSE13213 (n=79, HR=5.31 [1.99-14.2], p=0.00020) (FIG. 3B).

The 18-Hub-Gene Prognostic Signature is ADC-Specific.

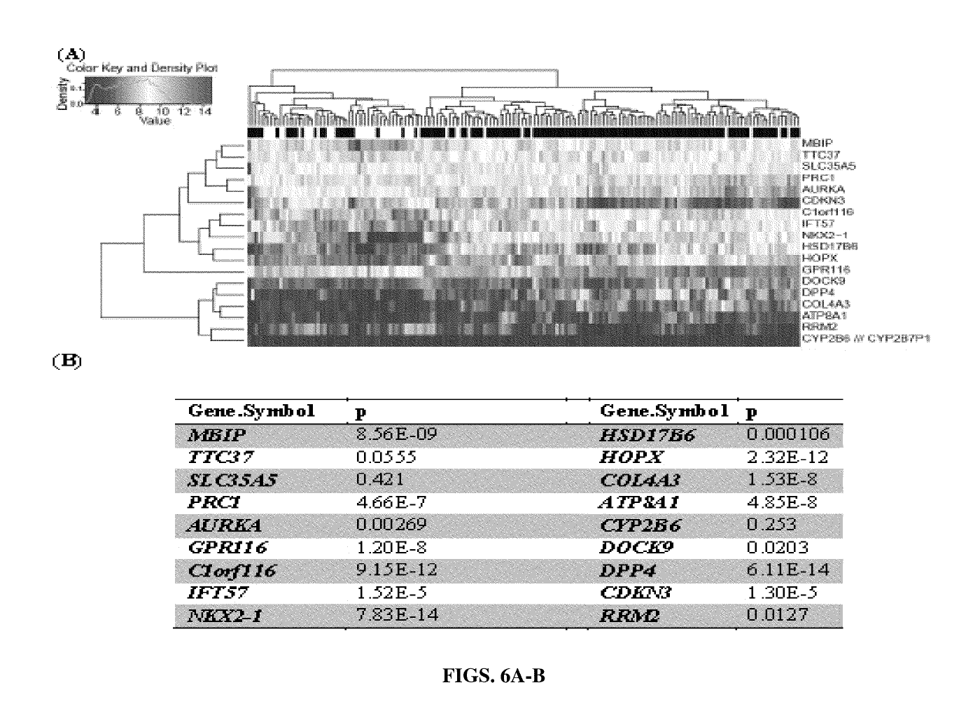

ADC and SCC are two major NSCLC histology subtypes with fundamentally different molecular makeup (40). Since the 18-hub-gene prognostic signature was derived from a cohort of ADC patients only, the inventors wanted to determine whether it was specific for ADC or could also predict prognosis for SCC patients. They tested the 18-hub-gene prognostic signature in SCC patients from GSE3141 (n=53), UT Lung SPORE (n=33), GSE8894 (n=76), GSE11969 (n=35), GSE4573 (n=129). The results (FIG. 3C) show that the signature does not predict survival in any of the 5 datasets. Note that 4 datasets (GSE3141, SPORE, GSE8894 and GSE13213) have both ADC and SCC patients, and the 18-hub-gene signature has significant prognostic values in all ADC sub-cohorts, but not in any SCC sub-cohorts. These results show that the 18-hub-gene prognostic signature is ADC-specific (p=0.00047 for interaction between histology and signature). In addition, 15 out of the 18 hub genes express differently between ADC and SCC patients, and unsupervised clustering analysis based on the expression of the 18 hub genes divided the patients into an ADC dominated group and a SCC dominated group (FIGS. 6A-B).

The 18-Hub-Gene Set has Better Performance than Top-Ranked Genes.

Selecting an optimal small set of genes from a large candidate gene list is a critical step for developing clinically practical molecular assays. The most widely used ranking based approaches (10) select genes with the most prominent p values obtained from individual gene-based testing. The inventors derived an 18-top-ranked-gene set containing 18 genes with the most significant association with the survival outcome based on the multivariate Cox model adjusted for age, cancer stage, and sample processing sites using the Consortium dataset. Here, they compared the performance of the 18-hub-gene set with the 18-top-ranked-gene set and the whole 797 survival related gene set (797-SR-gene set), all derived from the Consortium dataset.

Comparing the Prognostic Performances.

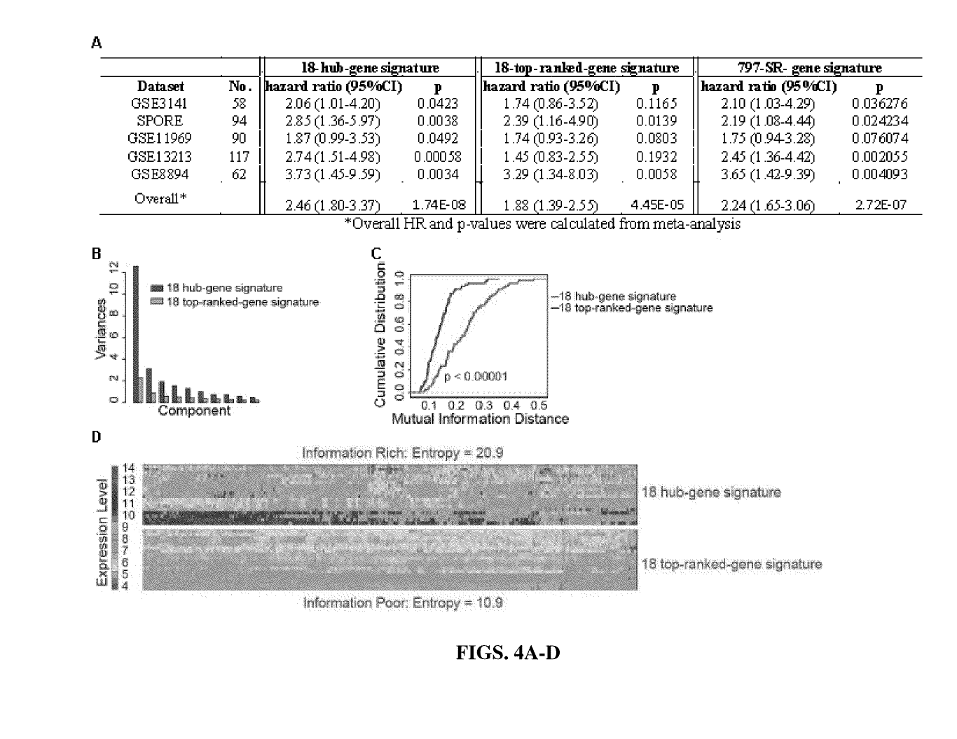

Using the Consortium dataset as the training set, the prognosis performances of the 18-hub-gene set, 18-top-ranked-gene set and 797-SR-gene set were compared in 5 independent validation sets for ADC patients. FIG. 4A shows that the 18-hub-gene signature consistently predicted prognosis in all 5 validation sets (HR=2.46, p=1.74E-08 from meta-analysis), and outperformed the 18-top-ranked-gene signature (HR=1.88, p=4.45E-05 from meta-analysis), which predicted prognosis (with p value <0.05) in only 2 out of 5 datasets. Furthermore, the 18-hub-gene signature has similar or even better prognostic performance than the 797-SR-gene signature (HR=2.24, p=2.72E-07) (FIG. 4A). It suggests that the hub-gene approach can effectively reduce the number of genes in the signature without sacrificing the prediction performance.

Comparing the Information Content.

The inventors used information theory approach (see supplementary methods) to study the reason why the hub-gene approach works well. The 18-hub-gene set has significantly higher pair-wise mutual information distance (a measure for independency) than the 18-top-ranked-gene set (p=1E-9, FIG. 4B), indicating that the hub-gene set has lower information redundancy than the top-ranked-gene set. As a result, the 18-hub-gene set has much higher entropy (a measure for information content, FIG. 4D) and captures more variation across patient population (FIG. 4C) than the 18-top-ranked-gene set. In summary, the hub-gene approach can effectively retain information while largely reducing the number of genes in the signature, which is important for developing clinically practical assays.

Derivation of a 12-Gene Set.

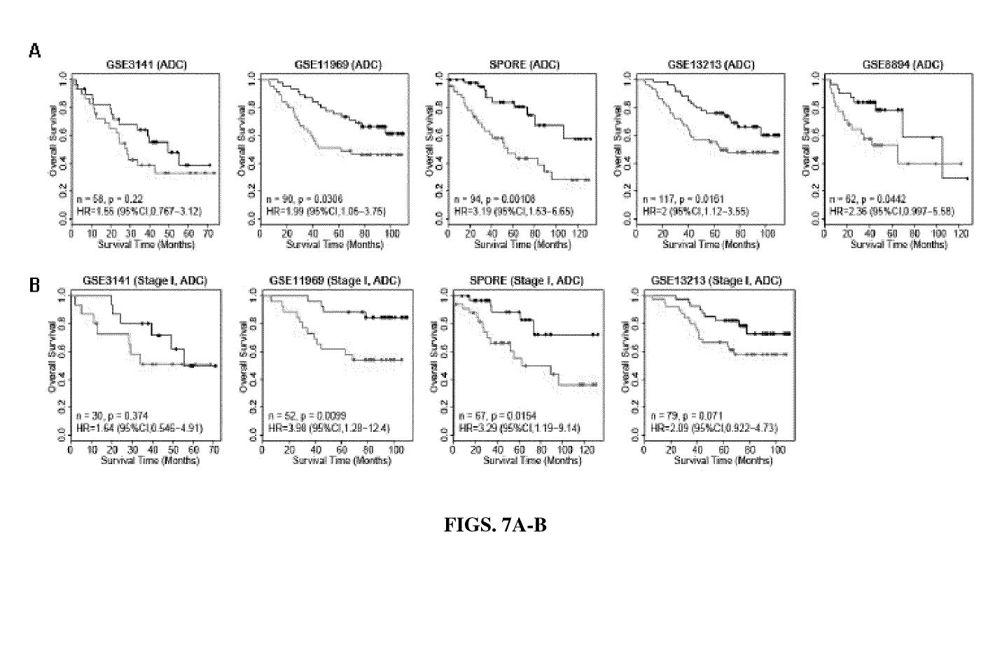

FIG. 1B illustrates the procedures for deriving and validating the 12-gene signature. First, the inventors found that 7 out of the 18 hub genes have significant genetic aberration in lung cancer using the Tumorscape program (world-wide-web at broadinstitute.org/tumorscape) (FIG. 2B), including a key lung cancer driver gene (NKX2-1) (Weir et al., 2007). Furthermore, 9 out of the 18 hub genes were “synthetic lethal” with paclitaxel for NSCLC (i.e., siRNA gene-specific knockdowns which killed NSCLC cells only in the presence of paclitaxel) based on the inventors' previous study (Whitehurst et al., 2007) (FIG. 2B). In total, 12 out of 18 hub genes either have genetic aberration or are ‘synthetic lethal’ for paclitaxel in lung cancer. These genes are DOCK9, RRM2, AURKA, HOPX, NKX2-1, TTC37, COL4A3, IFT57, Clorf116, HSD17B6, MBIP, and ATP8A1. Among these 12 genes, the expression of RRM2 and AURKA are associated with poor prognosis, and all other 10 genes are associated with good prognosis. The inventors developed a prediction model (12-gene signature) using the expression of these 12 genes and patients' survival outcomes in the Consortium dataset (training set) based on the superPC model and tested its prognostic effects on five independent ADC cohorts. The predicted high-risk group has significantly worse survival outcomes than the predicted low-risk group in the testing cohorts: UT Lung SPORE (n=94, HR=3.19 [1.53-6.65], p=0.00108), GSE8894 (n=62, HR=2.36 [1.00-5.58], p=0.0442), GSE3141 (n=58, HR=1.55 [0.77-3.12], p=0.22), GSE11969 (n=90, HR=1.99 [1.05-3.75], p=0.03), GSE13213 (n=117, HR=2.00[1.12-3.55], p=0.016) (FIG. 7A-B), the overall effect based on the meta-analysis (HR=2.10, p=1.79E-06).

The 12-Gene Signature Predicts Survival Benefits from ACT in NSCLC.

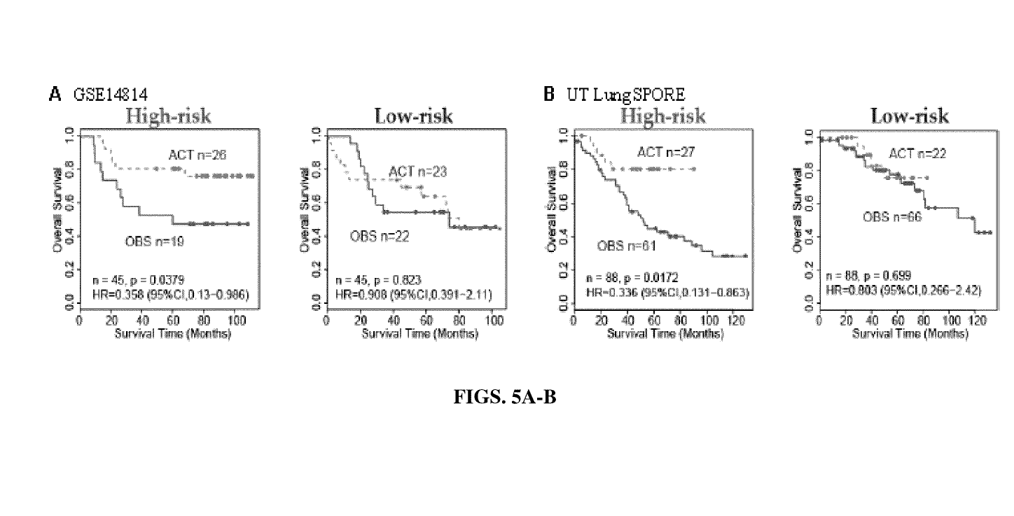

Because these 12 genes are “hubs” of the survival related genes, and play roles in cell response to chemotherapy drugs or have genetic aberrations in lung cancer, the inventors hypothesized that this 12-gene set can predict survival benefits from ACT in NSCLC. To test this hypothesis, they tested whether the 12-gene signature can predict which patients would benefit from ACT using two independent validation sets: (Jemal et al., 2008) 90 NSCLC samples from JBR.10 clinical trial (Zhu et al., 2010) in which 49 patients received vinorelbine plus cisplatin ACT treatment and 41 patients did not receive ACT; (Douillard et al., 2006) 176 NSCLC samples from UT Lung SPORE in which 49 patients received ACT (mainly Carboplatin plus Taxanes) and 127 patients did not receive ACT. Each patient in the validation sets was classified into a high- or low-risk group based on the 12-gene signature. Different from the prognosis biomarkers, no study has shown that the predictive biomarkers for chemotherapy are ADC- or SCC-specific. Therefore, the inventors tested the 12-gene signature in all NSCLC patients as other predictive biomarker studies (Chen et al., 2011; Zhu et al., 2010 and Olaussen et al., 2006). For the JBR.10 dataset, the ACT-treated patients showed longer survival than those without ACT (HR 0.36 [0.13-0.97], p=0.038; FIG. 5A) in the high-risk group; while patients with ACT treatment had no significant survival benefits (HR, 0.91[0.391-2.11], p=0.823; FIG. 5A) in the low-risk group. Furthermore, the patients with ACT treatment even have worse survival outcomes in the first 21 months for the low-risk group. The signature has a similar predictive effect in the UT Lung SPORE data: the patients who received ACT had better overall survival in the high-risk group (HR=0.34 [0.13-0.86], p=0.017, FIG. 5B), but not in the low-risk group (HR=0.80 [0.266-2.42], p=0.70, FIG. 5B).

### Example 3

### Discussion

This is the first study to use systems biology approaches to identify hub genes for prognostic and predictive signatures in lung cancer. Feature selection, which selects the most predictive genes while excluding the redundant genes to reduce the cost, is a critical step in developing a clinically practical molecular assay. A commonly used selection approach is based on ranking the performance of individual features (genes), and selecting the top ranked features. However, the combination of top ranked individual genes may not be optimal, because it does not consider relationship and potential information redundancy among genes.

In this study, the inventors applied a systems biology approach to identify hub genes which have 7-30 connections with other genes in the constructed survival-related network (FIGS. 2A-B), so the expression changes of these hub genes will affect many other genes and lead to substantial changes at the system level. This 18-hub-gene set has higher information content (FIGS. 4B-D) than the 18-top-ranked-gene set and has remarkably robust prognosis performances across different datasets and microarray platforms. From the Molecular Signatures Database (MsigDB), the inventors identified four lung cancer prognosis signatures derived from the same training dataset (the Consortium dataset). In addition, they identified another four NSCLC prognosis signatures with similar number of genes from the literature (Chen et al., 2007; Zhu et al., 2010; Ramaswamy et al., 2003; Bianchi et al., 2007). The inventors compared the prediction performances of the 18-hub-gene signature and the eight prognosis signatures in GSE13213 (n=117 for ADC) which has the most ADC patients in the testing datasets, and the 18-hub-gene signature clearly outperforms all other eight signatures (Table 3). These results indicate that the hub genes capture the key mRNA expression information related to NSCLC patients' survival.

The 18 hub genes, identified through a purely data-driven approach, have important biological implications in tumor development, including seven cancer metastasis genes and one key lung cancer driver gene (NKX2-1), demonstrating the biological relevance of this approach. To understand the potential biological and therapeutic relevance of the identified hub gene signature, the inventors downloaded all the gene lists from the MSigDB C2 gene sets database, and evaluated the overlap between these signatures and the gene lists (Table 4). Most notably, all of the hub genes have been identified in at least one gene list concerning cancer or carcinoma, while 7 genes are associated with cancer metastasis gene lists, and 6 genes are related to proliferation. The large overlap with cancer-associated gene lists implies that this prognostic gene signature is biologically relevant, and it is likely that the prognostic power is originated from their association with cancer metastasis or tumor cell proliferation. In particular, NKX2-1 and HOPX are important for the activation of p53 pathways and potentially helpful in repressing lung ADC development (Sweet-Cordero et al., 2005; Winslow et al., 2011), and could be promising candidates for lung cancer therapy. In addition, AURKA, PRC1, CDKN3, MBIP and RRM2 have all been reported to play important roles in tumor pathogenesis and are worth of further investigation.

This is also the first study to integrate RNAi functional screening data (Whitehurst et al., 2007) with mRNA expression and genetic aberration data (Weir et al., 2007 and Beroukhim et al., 2010) to identify a gene signature that predicting the benefits of ACT in lung cancer. Most of the current lung cancer gene signatures predict patients' outcomes irrespective of treatment (prognostic only). By contrast, the predictive signature developed from this study predicts the benefits of ACT in individual patients, and may have a direct impact on clinical decisions regarding which patients should get adjuvant chemotherapy (Arriagada et al., 2004). This 12-gene signature is predictive for ACT benefits in NSCLC for both paclitaxel or vinorelbine plus cisplatin (JBR.10 clinical trial cohort), and commonly used combinations such as carboplatin plus taxanes (UT Lung SPORE cohort). In addition to the prediction performance of molecular signatures, the biological relevance of genes in the signature is equally important. In this study, the hub genes were integrated with genes that are “synthetic lethal” with paclitaxel for NSCLC together with lung cancer genetic alternation information to identify a 12-gene functional set, which could be of great therapeutic importance as well as serve as a predictive biomarker. In particular, Aurora A kinase (AURKA) is associated with poor prognosis (HR=1.22, p=0.0061), connects with 12 survival associated genes, and is “synthetic lethal” with paclitaxel in NSCLC. AURKA is a key mitotic regulator and a target for anticancer drug. Currently, several small-molecule inhibitors of Aurora kinases are undergoing clinical evaluation for different types of cancer (Matulonis et al., 2012; Shan et al., 2012). All these suggest that AURKA may serve as both a predictive marker and a therapeutic target in NSCLC. In addition, the EGFR mutation and ALK rearrangement could be important to patient response to chemotherapy, and further studies are need to test how these mutations could affect the usage of the 12-gene signature. The 12-gene signature is both prognostic for ADC patients and predictive for adjuvant chemotherapy, so this signature has the potential to facilitate clinical decisions on using adjuvant chemotherapy for early stage NSCLC patients. On the other hand, the 18-gene set is a strong prognostic signature for early stage ADC patients, so if the goal is to predict patients' prognosis only, the 18-gene signature could be very helpful.

Although the current study shows the promising results and interesting functional relevance of the 12-gene signature, one limitation of this study is that the sample size is not big enough to test the interaction between the signature and the treatment groups. Thousands of patients are required to test the interaction between the predictive markers and treatment groups (Sargent et al., 2005), and no existing lung cancer genomic study can reach such sample size requirement. Since the long-term survival outcome may be confounded by other non-treatment factors, the inventors tested the interaction between signature groups and treatment using the survival in first three years after treatment. For JBR10 data, the interaction between the 12-gene signature and the treatment groups is significant (p=0.0005). The SPORE testing data is from a retrospective study. This dataset has limited sample size with treatments and the follow-up time is short, so the number of observed events is too small to reach the significant p value for the interaction term. Therefore, a further prospective study with large sample size is needed to valid the 12-gene signature as a predictive signature.

Because of the complexity of analytic procedures in the development of molecular signatures, it is essential to have rigorous validation in independent datasets/cohorts (Subramanian and Simon, 2010) in order to avoid potential over-fitting problems. In this study, the inventors validated the 18-hub-gene prognosis signature in 6 independent datasets across five different microarray platforms (including Affymetrix U133Plus2, Affymetrix U133A, Illumina Human-6 V3, Agilent 21.6K custom arrays, and Agilent 44K), and the validation cohorts include three studies conducted in western countries and three studies conducted in Asia. The prognosis performances are consistent across these heterogeneous populations and experimental techniques. The inventors tested the 12-gene predictive signature in two independent cohorts: the JBR.10 clinical trial and the UT Lung SPORE cohort. To the inventors' knowledge, this is the first study to include two validation datasets for predictive signatures in lung cancer. Zhu et al. (17) and Chen et al. (8) developed a predictive signature for ACT lung cancer, but it was only tested on the JBR.10 trial data. The UT Lung SPORE cohort used carboplatin plus taxanes based ACT treatments and the microarray experiment platform is different. All these results show the robustness of the prognosis and predictive signatures developed from this study. To facilitate other researchers to reproduce all results in this study, the inventors have provided a literate programming R package (SWEAVE report) in the supplementary material.

In summary, through systems biology approaches the inventors have identified a robust 18-hub-gene signature for prognosis of resected NSCLC patients. Furthermore, they developed a 12-gene prognostic and predictive signature for ACT benefit in NSCLC patients using integrative analysis approaches. A prospective clinical study is needed to further validate the clinical value of the prognosis and predictive signatures in the decision-making process of ACT for resected NSCLC patients.

TABLE 1
Meta-analysis of 18-hub-gene prognostic signature on ADC patients and
SCC patients. Data were summarized from 4 data sets that includes both
ADC and SCC patients: GSE13213, SPORE, GSE11969, and GSE8894

| | ADC patients | | SCC patients | |
|---|---|---|---|---|
| | hazard ratio (95% CI) | p | hazard ratio (95% CI) | p |
| Overall | 2.36 (1.64-3.41) | 4.51E−06 | 1.46 (0.98-2.18) | 6.45E−02 |
| Overall_clinical | 1.77 (1.15-2.74) | 9.60E−03 | 1.57 (0.93-2.66) | 8.90E−02 |
| variable adjusted | | | | |

TABLE 2
Multivariate Cox regression analysis of the 18-hub-gene prognostic
signature and clinical variables in the UT Lung SPORE

| | | Hazard Ratio (95% CI) | p |
|---|---|---|---|
| Risk groups predicted | high vs. low | 2.93 (1.25-6.88) | 0.0137 |
| from the 18-hub-gene | | | |
| signature | | | |
| Age | | 1.05 (1.00-1.09) | 0.0309 |
| Stage | II vs. I | 0.82 (0.35-1.92) | 0.6432 |
| | III vs. I | 1.34 (0.49-3.70) | 0.5709 |
| Gender | M vs. F | 0.94 (0.44-2.00) | 0.8651 |

TABLE 3
Comparison with the gene signatures with other signatures

| | No. of Genes | Hazard Ratio (95% CI) | p | Reference |
|---|---|---|---|---|
| 18-hub-gene signature | 18 | 2.74 (1.51-4.98) | 0.00058 | |
| GOOD_SURVIVAL_A4¹ | 199 | 1.89 (1.06-3.35) | 0.0278 | (13) |
| GOOD_SURVIVAL_A5² | 70 | 1.47 (0.84-2.60) | 0.1762 | (13) |
| POOR_SURVIVAL_A6³ | 459 | 2.28 (1.26-4.10) | 0.00486 | (13) |
| GOOD_SURVIVAL_A12⁴ | 348 | 1.62 (0.92-2.86) | 0.0910 | (13) |
| Bianchi | 10 | 1.95 (1.10-3.47) | 0.0205 | (43) |
| Chen | 5 | 1.83 (1.03-3.26) | 0.0358 | (9) |
| Zhu | 15 | 2.11 (1.18-3.77) | 0.0103 | (17) |
| Ramaswamy | 17 | 2.03 (1.13-3.63) | 0.0150 | (42) |

¹Cluster 4 of method A: up-regulation of these genes in patients with NSCLC predict good survival outcome. http://www.broadinstitute.org/gsea/msigdb/cards/SHEDDEN_LUNG_CANCER_GOOD_SURVIVAL_A4.html
²Cluster 5 of method A: up-regulation of these genes in patients with NSCLC predict good survival outcome. http://www.broadinstitute.org/gsea/msigdb/cards/SHEDDEN_LUNG_CANCER_GOOD_SURVIVAL_A5.html
³Cluster 6 of method A: up-regulation of these genes in patient with NSCLC predicts poor survival outcome. http://www.broadinstitute.org/gsea/msigdb/cards/SHEDDEN_LUNG_CANCER_GOOD_SURVIVAL_A6.html
⁴Cluster 12 of method A: up-regulation of these genes in patients with NSCLC predicts good survival outcome. http://www.broadinstitute.org/gsea/msigdb/cards/SHEDDEN_LUNG_CANCER_GOOD_SURVIVAL_A12.html

From the Molecular Signatures Database (MsigDB), the inventors identified 4 other signatures derived from the same Consortium training dataset by Shedden et al. (2008). As demonstrated by the Shedden et al., signatures derived by method A had the best overall performance. The signatures named as cluster 4, 5, 6, and 12 are the gene clusters from method A gene set, consisting of genes with correlated expressions. The details of the clusters are discussed in the original paper. In addition, the inventors identified another four NSCLC prognosis signatures with similar number of genes from the literatures (Chen et al., 2007; Zhu et al., 2010; Ramaswamy et al., 2003; Bianchi et al., 2007). The inventors compared the prognostic performance of the 18-hub-gene signature and the other eight signatures. The Consortium dataset was used as training set and the GSE13213 (n=117 for ADC) was used as test set. Hazard ratios and p-values were derived from Cox-regression models comparing the predicted high vs. low risk groups. The 18-hub-gene signature outperforms other signatures.

TABLE 4
Members of the 18-hub gene signature have been
included in MSigDB database C2 signature catalogues

| | Overlapping | | | | | | | | | | | |
|---|---|---|---|---|---|---|---|---|---|---|---|---|
| | MSigDB | | Metas- | Prolif- | | | | | Thy- | | | |
| Genes | c2 gene list | Cancer | tasis | eration | Lung | Breast | Liver | Lymphasyte | roid | KEGG | BIOCARTA | REACTOME |
| RRM2 | 123 | 15 | 3 | 2 | 1 | 5 | | 7 | 1 | 4 | | 6 |
| AURKA | 111 | 18 | 4 | 2 | 1 | 8 | 2 | 4 | 2 | 1 | 1 | 1 |
| CDKN3 | 81 | 12 | 1 | 2 | 1 | 2 | 4 | 6 | | | | |
| PRC1 | 67 | 18 | 2 | 3 | 1 | 6 | 3 | 4 | 2 | | | |
| HOPX | 31 | 4 | 1 | | 2 | 1 | | | | | | |
| DPP4 | 29 | 9 | 1 | | | 1 | 7 | | 3 | | | |
| ATP8A1 | 26 | 4 | | | 1 | 5 | | 1 | 1 | | | |
| CYP2B6 | 25 | 12 | | 1 | 1 | 6 | 3 | | | 4 | | 4 |
| DOCK9 | 23 | 2 | | | | | | | 1 | | | |
| COL4A3 | 18 | 2 | | | | | | | 1 | | 5 | 5 |
| C1of116 | 18 | 6 | 1 | | 1 | 2 | | | | | | |
| TTC37 | 17 | 4 | | | | | 2 | | 1 | 1 | | |
| IFT57 | 16 | 3 | | | | 1 | | | | | | |
| HSD17B6 | 15 | 3 | | 1 | | 1 | 1 | | 1 | 1 | | |
| NKX2-1 | 15 | 6 | | | 2 | | | | 2 | | | |
| GPR116 | 13 | 10 | | | 1 | 3 | 4 | | | | | |
| MBIP | 7 | 3 | | | 2 | | 1 | | | | | |
| SLC35A5 | 5 | 1 | | | | | | | | | | |
| Grand | 654 | 143 | 13 | 11 | 15 | 46 | 30 | 22 | 15 | 12 | 6 | 16 |
| Total | | | | | | | | | | | | |

To understand the potential biological and therapeutic relevance of the identified hub gene signature, the inventors downloaded all the gene lists from the MSigDB C2 database (world-wide-web at broadinstitute.org/gsea/msigdb), and evaluated the overlap between our signatures and the gene lists. Most notably, all of the hub genes have been identified in at least one gene list concerning cancer or carcinoma, while 7 out of 19 are associated with cancer metastasis gene lists, and 6 are related to proliferation. The large overlap with cancer associated gene lists implies that the 18 hub genes are biologically relevant.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### V. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,415,723
U.S. Pat. No. 4,458,066
U.S. Pat. No. 4,683,195
U.S. Pat. No. 4,683,202
U.S. Pat. No. 4,800,159
U.S. Pat. No. 4,883,750
U.S. Pat. No. 5,143,854
U.S. Pat. No. 5,202,231
U.S. Pat. No. 5,242,974
U.S. Pat. No. 5,279,721
U.S. Pat. No. 5,288,644
U.S. Pat. No. 5,324,633
U.S. Pat. No. 5,384,261
U.S. Pat. No. 5,405,783
U.S. Pat. No. 5,412,087
U.S. Pat. No. 5,424,186
U.S. Pat. No. 5,429,807
U.S. Pat. No. 5,432,049
U.S. Pat. No. 5,436,327
U.S. Pat. No. 5,445,934
U.S. Pat. No. 5,468,613
U.S. Pat. No. 5,470,710
U.S. Pat. No. 5,472,672
U.S. Pat. No. 5,492,806
U.S. Pat. No. 5,503,980
U.S. Pat. No. 5,510,270
U.S. Pat. No. 5,525,464
U.S. Pat. No. 5,527,681
U.S. Pat. No. 5,529,756
U.S. Pat. No. 5,532,128
U.S. Pat. No. 5,545,531
U.S. Pat. No. 5,547,839
U.S. Pat. No. 5,554,501
U.S. Pat. No. 5,556,752
U.S. Pat. No. 5,561,071
U.S. Pat. No. 5,571,639
U.S. Pat. No. 5,580,726
U.S. Pat. No. 5,580,732
U.S. Pat. No. 5,593,839
U.S. Pat. No. 5,599,672
U.S. Pat. No. 5,599,695
U.S. Pat. No. 5,610,287
U.S. Pat. No. 5,624,711
U.S. Pat. No. 5,631,134
U.S. Pat. No. 5,639,603
U.S. Pat. No. 5,654,413
U.S. Pat. No. 5,658,734
U.S. Pat. No. 5,661,028
U.S. Pat. No. 5,665,547
U.S. Pat. No. 5,667,972
U.S. Pat. No. 5,695,940
U.S. Pat. No. 5,700,637
U.S. Pat. No. 5,739,169
U.S. Pat. No. 5,744,305
U.S. Pat. No. 5,795,715
U.S. Pat. No. 5,800,992
U.S. Pat. No. 5,801,005
U.S. Pat. No. 5,807,522
U.S. Pat. No. 5,824,311
U.S. Pat. No. 5,830,645
U.S. Pat. No. 5,830,880
U.S. Pat. No. 5,837,196
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,846,945
U.S. Pat. No. 5,847,219
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,871,928
U.S. Pat. No. 5,876,932
U.S. Pat. No. 5,882,864
U.S. Pat. No. 5,889,136
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 6,004,755
U.S. Pat. No. 6,087,102
U.S. Pat. No. 6,368,799
U.S. Pat. No. 6,383,749
U.S. Pat. No. 6,506,559
U.S. Pat. No. 6,573,099
U.S. Pat. No. 6,617,112
U.S. Pat. No. 6,638,717
U.S. Pat. No. 6,720,138
U.S. Patent Publn. 2002/0168707
U.S. Patent Publn. 2003/0051263
U.S. Patent Publn. 2003/0055020
U.S. Patent Publn. 2003/0159161
U.S. Patent Publn. 2004/0064842
U.S. Patent Publn. 2004/0265839
U.S. Patent Publn. 2008/0009439
Abbondanzo et al., Breast Cancer Res. Treat., 16:182(151), 1990.
Allen et al., Briefings in bioinformatics, 13:547-54, 2012.
Allred et al., Arch. Surg., 125(1):107-113, 1990.
Arriagada et al., N Engl J Med, 350:351-60, 2004.
Austin-Ward and Villaseca, Revista Medica de Chile, 126(7):838-845, 1998.
Bair and Tibshirani, PLoS Biol, 2:E108, 2004.
Bellus, J. Macromol. Sci. Pure Appl. Chem., A31(1): 1355-1376, 1994.
Beroukhim et al., Nature, 463:899-905, 2010.
Bianchi et al., J Clin Invest, 117:3436-44, 2007.
Bild et al., Nature, 439:353-7, 2006.
Boutros et al., Proc Natl Acad Sci USA, 106:2824-8, 2009.
Brown et al. Immunol. Ser., 53:69-82, 1990.
Breiman et al., Chapman & Hall/CRC 1984.
Bukowski et al., Clinical Cancer Res., 4(10):2337-2347, 1998.
Capaldi et al., Biochem. Biophys. Res. Comm., 74(2):425-433, 1977.
Chen et al., N Engl J Med, 356:11-20, 2007.
Chen et al., J Natl Cancer Inst, 103:1859-70, 2011.
Christodoulides et al., Microbiology, 144(Pt 11):3027-3037, 1998.
Collett, Chapman & Hall/CRC 2003.
Davidson et al., J. Immunother., 21(5):389-398, 1998.
De Jager et al., Semin. Nucl. Med., 23(2):165-179, 1993.
Douillard et al., Lancet Oncol, 7:719-27, 2006.
Frohman, In: PCR Protocols: A Guide To Methods And Applications, Academic Press, N.Y., 1990.
GB Application No. 2 202 328
Hacia et al., Nat. Genet., 14:441-449, 1996.
Hanibuchi et al., Int. J. Cancer, 78(4):480-485, 1998.
Hellstrand et al., Acta Oncologica, 37(4):347-353, 1998.
Herbst et al., N Engl J Med, 359:1367-80, 2008.
Hui and Hashimoto, Infection Immun., 66(11):5329-5336, 1998.
Innis et al., Proc. Natl. Acad. Sci. USA, 85(24):9436-9440, 1988.
Irizarry et al., Nucleic Acids Res, 31:e15, 2003.
Jemal et al., CA Cancer J Clin, 58:71-96, 2008.
Jeong et al., PLoS Med, 7:e1000378, 2010.
Ju et al., Gene Ther., 7(19):1672-1679, 2000.
Kaplan, Journal of the American Statistical Association, 457-81, 1958.
Kato et al., N Engl J Med, 350:1713-21, 2004.
Kratz et al., Lancet, 379:823-32, 2012.
Kwoh et al., Proc. Natl. Acad. Sci. USA, 86:1173, 1989.
Lee et al., Clin Cancer Res, 14:7397-404, 2008.
Lu et al., PLoS Med, 3:e467, 2006.
MacBeath and Schreiber, Science, 289(5485):1760-1763, 2000.
Maitra et al., Curr Mol Med, 1:153-62, 2001.
Matsuyama et al., Mol Carcinog, 50:301-9, 2011.
Matulonis et al., Gynecol Oncol, 2012.
Mitchell et al., Ann. NY Acad. Sci., 690:153-166, 1993.
Mitchell et al., J. Clin. Oncol., 8(5):856-869, 1990.
Morton et al., Arch. Surg., 127:392-399, 1992.
Nakamura et al., In: Handbook of Experimental Immunology (4^{th} Ed.), Weir et al. (Eds), 1:27, Blackwell Scientific Publ., Oxford, 1987.
Navab et al., Proc Natl Acad Sci USA, 108:7160-5, 2011.
Ohara et al., Proc. Natl. Acad. Sci. USA, 86:5673-5677, 1989.
Olaussen et al., N Engl J Med, 355:983-91, 2006.
Olaussen et al., Curr Opin Pulm Med, 13:284-9, 2007.
Pandey and Mann, Nature, 405(6788):837-846, 2000.
Pease et al., Proc. Natl. Acad. Sci. USA, 91:5022-5026, 1994.
Peng et al., Journal of the American Statistical Association, 104:735-46, 2009.
Pietras et al., Oncogene, 17(17):2235-2249, 1998.
Pounds and Morris, Bioinformatics, 19:1236-42, 2003.
Qin et al., Proc. Natl. Acad. Sci. USA, 95(24):14411-14416, 1998.
Raponi et al., Cancer Res, 66:7466-72, 2006.
Ramaswamy et al., Nat Genet, 33:49-54, 2003.
Ravindranath and Morton, Intern. Rev. Immunol., 7: 303-329, 1991.
Roepman et al., Clin Cancer Res, 15:284-90, 2009.
Rosenberg et al., Ann. Surg. 210(4):474-548, 1989.
Rosenberg et al., N. Engl. J. Med., 319:1676, 1988.
Sargent et al. J Clin Oncol, 23:2020-7, 2005.
Schwarzer, Meta: Meta-analysis with r. 2012.
Shan et al., Clin Cancer Res, 18:3352-65, 2012.
Shedden et al., Nature Medicine, 14:822-7, 2008.
Shoemaker et al., Nature Genetics, 14:450-456, 1996.
Subramanian and Simon, J Natl Cancer Inst, 2010.
R. Simon, British J. Cancer 89, 1599-1604, 2003.
Strauss et al., J Clin Oncol, 26:5043-51, 2008.
Sweet-Cordero et al., Nat Genet, 37:48-55, 2005.
The International Adjuvant Lung Cancer Trial Collaborative Group., N Engl J Med, 350:351-60, 2004.
Tomida et al., Oncogene, 23:5360-70, 2004.
Tomida et al., J Clin Oncol, 27:2793-9, 2009.
Walker et al., Nucleic Acids Res. 20(7):1691-1696, 1992.
Weir et al., Nature, 450:893-8, 2007.
Whitehurst et al., Nature, 446:815-9, 2007.
Wigle et al., Cancer Res, 62:3005-8, 2002.
Winslow et al., Nature, 473:101-4, 2011.
Winton et al., N Engl J Med, 352:2589-97, 2005.
Xie et al., Bioinformatics, 25:751-7, 2009.
Xie et al., Clin Cancer Res, 17:5705-14, 2011.
Zhu et al., J Clin Oncol, 28:4417-24, 2010.

1. A method of predicting the survival of a human subject diagnosed with carcinoma tumor of the lung comprising obtaining expression information for 9 or more of the following genes in a lung carcinoma cancer sample obtained from said subject:
RPM2, ARUKA, CDKN3, PRC1, HOPX, DPP4, ATP8A1, CYP2B6, DOCKS, COL4A3, Clorf116, TTC37, IFT57, HSD17B6, NKX2-1, GPR116, MBIP, and/or SLC35A5,
wherein an alteration in the expression 9 or more of said genes, as compared to the average expression in an carcinoma of the lung, indicates that said subject has a worse than average survival.

2. The method of claim 1, wherein the decrease and/or increase of expression is at least 0.2-fold.

3. The method of claim 1, further comprising treating said patient with an aggressive therapy if predicted to have a worse than average prognosis for survival.

4. The method of claim 1, wherein obtaining expression information comprises assessing protein expression.

5. (canceled)

6. The method of claim 1, wherein obtaining expression information expression comprises assessing mRNA expression.

7-9. (canceled)

10. The method of claim 1, further comprising resecting said tumor.

11. The method of claim 3, wherein aggressive therapy comprises chemotherapy and/or radiotherapy.

12. The method of claim 11, wherein chemotherapy comprises a platin compound and/or a taxane compound.

13. The method of claim 1, further comprising obtaining expression information for 10, 11, 12, 13, 14, 15, 16, 17, or all 18 of said genes.

14. The method of claim 1, wherein said carcinoma of the lung is early stage adenocarcinoma.

15. The method of claim 1, wherein the expression of each of the following genes is assessed:
RPM2, ARUKA, HOPX, ATP8A1, DOCK9, COL4A3, Clorf116, TTC37, IFT57, HSD17B6, NKX2-1 and MBIP.

16. A method of predicting the chemotherapeutic response of a human subject diagnosed with a non-small cell lung cancer tumor comprising obtaining expression information for 6 or more of the following genes in a non-small cell lung cancer sample obtained from said subject:
RPM2, ARUKA, HOPX, ATP8A1, DOCK9, COL4A3, Clorf116, TTC37, IFT57, HSD17B6, NKX2-1 and/or MBIP,
wherein an alteration in the expression 6 or more of said genes, as compared to the average expression in non-small cell lung cancer, indicates that said subject will respond favorably to adjuvant chemotherapy.

17. The method of claim 1, wherein the decrease and/or increase of expression is at least 0.2-fold.

18. The method of claim 1, further comprising treating said patient with adjuvant chemotherapy if predicted to be a responder.

19. The method of claim 1, wherein obtaining expression information comprises assessing protein expression.

20. (canceled)

21. The method of claim 16, wherein obtaining expression information expression comprises assessing mRNA expression.

22-24. (canceled)

25. The method of claim 16, further comprising resecting said tumor.

26. (canceled)

27. The method of claim 16, further comprising obtaining expression information for 7, 8, 9. 10, 11, or all 12 of said genes.

28. The method of claim 16, further comprising treating said subject with a non-chemotherapy cancer treatment if predicted to be a non-responder.

29. (canceled)

30. The method of claim 1, wherein said subject has a stage I-III adenocarcinoma of the lung.
